(19) United States
(12) Patent Application Publication
(10) Pub. No.: US 2023/0310479 A1
(43) Pub. Date: Oct. 5, 2023

(54) LOW DOSE ADJUVANT EPIGENETIC CANCER THERAPY

(71) Applicant: THE JOHNS HOPKINS UNIVERSITY, Baltimore, MD (US)

(72) Inventors: Malcolm V. Brock, Owings Mills, MD (US); Zhihao Lu, Baltimore, MD (US)

(21) Appl. No.: 17/771,754

(22) PCT Filed: Oct. 26, 2020
(86) PCT No.: PCT/US2020/057316

§ 371 Date: Apr. 25, 2022

Related U.S. Application Data
(60) Provisional application No. 62/925,398, filed on Oct. 24, 2019.

(51) Int. Cl. A61K 31/706; A61P 35/00; A61K 31/4406

(52) U.S. Cl. CPC A61K 31/706; A61K 31/4406; A61P 35/00

(57) ABSTRACT

The present invention relates to the field of cancer. More specifically, the present invention provides compositions and methods useful for preventing tumor recurrence and metastases. In one embodiment, a method for inhibiting lung cancer metastases in a post-resection patient comprises the step of administering low dose adjuvant epigenetic therapy (LDAET) to inhibit the migration of myeloid-derived suppressor cells from the bone marrow to the lung premetastatic environment.

### CROSS-REFERENCE TO RELATED APPLICATIONS

This application claims the benefit of U.S. Provisional Application No. 62/925,398, filed Oct. 24, 2019, which is incorporated herein by reference in its entirety.

### FIELD OF THE INVENTION

The present invention relates to the field of cancer. More specifically, the present invention provides compositions and methods useful for preventing tumor recurrence and metastases.

### BACKGROUND OF THE INVENTION

Despite advances in traditional cancer treatments and newly developed immunotherapies, cancer recurrence after surgery of non-small-cell lung cancer (NSCLC), oesophageal cancer and breast cancer remains high at 35-76%^{2,8}, 30-66%^{9,10} and 20-66%³, respectively. Most cancer-related mortalities after resection are due to metastases.^{1,2} In early-stage NSCLC in particular, 30-55% of patients die from recurrent metastatic disease after surgery with curative intent^{8,11}, and standard adjuvant chemotherapy confers an absolute 5-year survival benefit of only 3-10%.¹¹ Current immune-directed neoadjuvant therapies using immune checkpoint blockade rely on the presence of the primary tumor to generate tumor-antigen-specific T cell responses,¹² but in clinical practice the majority of early-stage NSCLC tumors are removed without neoadjuvant intervention. ^{11,13} Novel strategies are needed that decrease tumor recurrence and metastases in the absence of a primary tumor.

### SUMMARY OF THE INVENTION

The present invention is based, at least in part, on the discovery that after surgical removal of a primary tumor, adjuvant epigenetic therapy can disrupt the pre-metastatic microenvironment and inhibit cancer recurrence by its selective effect on myeloid derived suppressor cells (MDSCs). As described herein, the present invention discloses that adjuvant epigenetic therapy, using low dose DNA methyltransferase and histone deacetylase inhibitors such as 5-azacytidine (AZA) and entinostat, disrupt the formation of pre-metastatic niches, by inhibiting the trafficking of MDSCs through the downregulation of CCR2 and CXCR2 and by promoting MDSCs differentiation into interstitial macrophages. Additional embodiments of the present invention include the administration of CCR2 and/or CXCR2 inhibitors. Decreased accumulation of MDSCs in the premetastatic lung resulted in longer, disease-free and overall survival compared to chemotherapy in mouse models. The present data demonstrated that even after complete surgical resection of a primary tumor, MDSCs can still contribute to the development of pulmonary premetastatic niches and settlement of residual tumor cells.

Accordingly, in one aspect, the present invention provides compositions and methods for inhibiting lung cancer metastases in a post-resection patient. In one embodiment, a method comprises the step of administering low dose adjuvant epigenetic therapy (LDAET) to inhibit the migration of myeloid-derived suppressor cells from the bone marrow to the lung premetastatic environment. In particular embodiments, LDAET comprises at least one epigenetic therapeutic agent including demethylating agents and histone deacytelase (HDAC) inhibitors. In certain embodiments, the LDAET comprises a demethylating agent. In specific embodiments, the demethylating agent comprises at least one of 5-azacytidine (azacytidine; AZA), 5-azadeoxycytidine (decitabine; DAC), SGI-110 (guadecitabine), zebularine or procaine.

In further embodiments, nucleoside analogs that target DNA methyltransferases (DNMTs) can be used. In addition to AZA and Decitabine, other nucleoside analogs that target DNMTs can be used including, but not limited to, 6-Thioguanine (Thioguanine, 6tG) (2-amino-1,7-dihydro-6h-purine-6-thione); 5-Fluoro-2′-deoxycytidine (FdCyd), pseudocytidine (ΨICyd) (2-amino-5-β-D-ribofuranosylpyrimidin-4(1H)-one); 5,6-Dihydro-5-azacytidine (DHAC); fazarbine (Ara-AC) (1-β-D-arabinofuranosyl-5-azacytosine); zebularine (Zeb) (1-(β-D-ribofuranosyl)-1,2 dihydropyrimidin-2-one); 2′-Deoxy-5,6-dihydro-5-azacytidine (DHDAC, KP-1212); 4′-Thio-2′-deoxycytidine (TdCyd); and 5-aza-4′-thio-2′-deoxycytidine (5-aza-TdCyd).

In other embodiments, prodrugs of AZA and decitabine can be used including, but not limited to, SGI-110, CP-4200 (5-azacytidine-5′-elaidate); RX-3117 (TV-1360, Fluorocyclopentenylcytosine); NPEOC-DAC (2′-deoxy-N4-[2-(4-nitrophenyl)ethoxycarbonyl]-5-azacytidine); and 2′3′ 5′Triacetyl-5-azacytidine (TAC).

In particular embodiments, the LDAET comprises administering an HDAC inhibitor. In specific embodiments, the HDAC inhibitor comprises at least one of givinostat, entinostat, trichostatin A (TSA), Vorinostat (SAHA), Valproic Acid (VPA), romidepsin (FK228, depsipeptide), MS-275, tucidinostat (chidamide), panobinostat (Farydak, LBH589), belinostat (PXD101), mocetinostat (MGCD0103), abexinostat (PCI-24781), SB939, resminostat (4SC-201),quisinostat (JNJ26481585), Kevetrin, CUDC-101, AR-42, CHR-2845, CHR-3996, Domatinostat (4SC-202), ricolinostat (ACY-1215), and ME-344.

Examples of HDAC inhibitors also include ACY-738, BG45, Biphenyl-4-sulfonyl chloride, BRD73954, CAY10603, Citarinostat (ACY-241), CUDC-907, Dacinostat (LAQ824), Droxinostat, ITSA-1 (ITSA1), LMK-235, M344, MC1568, Nexturastat A, PCI-34051, Pracinostat (SB939), RG2833 (RGFP109), RGFP966, Santacruzamate A (CAY10683), SKLB-23bb, Sodium butyrate, Splitomicin, Suberohydroxamic acid, Tacedinaline (CI994), Tasquinimod, TH34, Tinostamustine (EDO-S101), TMP195, TMP269, Tubacin, Tubastatin A, Tubastatin A HCl, UF010, and WT161.

In other embodiments, the method further comprises the step of administering a C-C chemokine receptor type 2 (CCR2) inhibitor. In specific embodiments, the CCR2 inhibitor comprises at least one of CCX140, CCX872, PF-04136309 (PF-6309), PF-04178903, INCB-8696, CCX-915, MLN-1202, JNJ-17166864; AZD-2423, INCB-003284, BMS-741672, MK-0812; PF-04634817, CNTO888, and 747 (kaempferol 3-(2,4-di-E-p-coumaroylrhamnoside).

In additional embodiments, the method further comprises the step of administering a C-X-C motif chemokine receptor 2 (CXCR2) inhibitor. In specific embodiments, the CXCR2 inhibitor comprises at least one of AZD5069, MK-7123 (SCH527123, Navarixin), SB-332235, danirixin, elubrixin, PS-291822, SB225002, SX-682, SX-576, SX-517, ladarixin, reparixin, reparixin L-lysine salt, DF2755A, CXCL8 fragment comprising amino acids 3-74 and substitutions K11R/G31P (G31P); DF2162 and SCH-479833.

In another aspect, the present invention provides compositions and methods for inhibiting cancer metastases in a post-resection patient. In one embodiment, a method comprises the step of administering LDAET to inhibit the migration of myeloid-derived suppressor cells from the bone marrow to the premetastatic environment. In particular embodiments, LDAET comprises at least one epigenetic therapeutic agent including demethylating agents and histone deacytelase (HDAC) inhibitors. In specific embodiments, the LDAET comprises nucleoside analogs that target DNMTs. In other embodiments, the method further comprises the step of administering a CCR2 inhibitor. In additional embodiments, the method further comprises the step of administering a CXCR2 inhibitor.

In other embodiments, a method for inhibiting cancer metastases in a post-resection patient comprises the step of administering LDAET to inhibit the migration of myeloid-derived suppressor cells from the bone marrow to the premetastatic environment.

### BRIEF DESCRIPTION OF THE FIGURES

FIGS. 1A-1F. Low-dose AET disrupts the lung premetastatic microenvironment by affecting MDSCs. FIG. 1A: Timeline of mouse models of metastasis with treatment schedules. CT, computed tomography. FIG. 1B: Longitudinal H&E and immunofluorescence staining of lung tissue showed the presence of tumor cells from day 6 in the LLC model. Immunofluorescence staining was performed using GFP (green) antibodies. Merged images contain 4′,6-diamidino-2-phenylindole (DAPI) DNA staining, which demarcates cell nuclei (blue). Scale bars, 2 mm. Graph shows the area and numbers of metastatic nodules (n=3 mice at each time point). Two-sample, two-sided t-test. FIG. 1C: Immune-cell profiles of lungs in the LLC model. Single-cell suspensions from both lungs were analyzed by fluorescence-activated cell sorting (FACS) (n=3 mice at each time point). NC, negative control (normal lungs from C57BL/6 mice). D, day. Two-sample, two-sided t-test was used in comparison with the negative control. T_{reg} cells, regulatory T cells. FIG. 1D: FACS showing lung MDSCs in the LLC model at day 3 were depleted using pep-H6 (leftmost two panels). Column diagram showing the effect of pep-H6 on the percentages of lung MDSCs and macrophages (middle two panels) at day 3 (n=3 mice in each group). Two-sample, two-sided t-test. Kaplan-Meier curves showing disease-free survival and overall survival of LLC mice after MDSC depletion (rightmost two panels). Two-sided log-rank test. Pep-irrel, irrelevant peptibody, used as control. FIG. 1E: Kaplan-Meier curves showing disease-free and overall survival of LLC mice after transfusion of lung MDSCs (5×10⁶) (leftmost two panels) and of monocytic (M-)MDSCs (5×10⁶) or polymorphonuclear (PMN-)MDSCs (5×10⁶)from bone marrow (rightmost two panels). All transfusions were conducted on day 1 and day 4. Two-sided log-rank test. FIG. 1F: FACS showing representative effects of low-dose (LD-)AET on lung MDSCs in LLC and HNM007 mice at day 3. Column diagrams showing the effect of low-dose AET on lung MDSCs in LLC and HNM007 mice (n=3 mice at each time point). Two-sample, two-sided t-test. Bars show mean ± s.e.m. NS, not significant, *P < 0.05, **P < 0.01, ***P < 0.001.

FIGS. 2A-2G. Low-dose AET inhibits migration of monocytic MDSCs from the bone marrow to the lung premetastatic microenvironment by downregulating expression of CCR2. FIGS. 2A, 2B: The effect of low-dose AET on monocytic MDSCs transferred from CD45.1 to CD45.2 mice (FIG. 2A) and the trafficking ability of adoptively transferred monocytic MDSCs from low-dose-AET-or vehicle-treated CD45.1 mice in CD45.2 recipient mice (FIG. 2B). FACS and graphs showing percentages and absolute numbers of the donor-derived-cell subset (CD45.1⁺ cells) in both lungs of the recipient LLC mice (n=3 mice per group). FIG. 2C: Cell-sorting schema for collecting monocytic MDSCs on day 3 after resection (left). Volcano plots showing differences in RNA expression of monocytic MDSCs from the bone marrow (BM) or lung between low-dose-AET-and vehicle-treated LLC mice (right) (n=3 biological replicates). FIG. 2D: Agilent cDNA array expression of chemokines and chemokine receptors in monocytic MDSCs from bone marrow or lungs of low-dose-AET-and vehicle-treated LLC mice (n=3 biological replicates). False-discovery-rate (FDR)-adjusted P values of CCR2 are 0.005 (bone marrow) and 0.016 (lung). Cxcr7 is also known as Ackr3. FIG. 2E: Effect of low-dose AET on Ccr2 expression in monocytic MDSCs from bone marrow (BM-M-MDSCs) from LLC mice on day 3, by quantitative PCR (left) (n=3 biological replicates) and FACS (right). In the FACS plot, red represents low-dose AET and blue represents mock treatment. FIG. 2F: Transwell migration assay of sorted monocytic MDSCs from bone marrow of low-dose-AET-or vehicle-treated LLC mice (day 3) induced by CCL2 for 60 min. Fold changes are normalized to the migration of the cells in the unstimulated mock group (set at 1) (n=3 biological replicates). FIG. 2G: Left, FACS showing representative results of lung MDSCs in Ccr2-knockout (KO) and wild-type (WT) LLC and HNM007 mice at day 3. Right, Kaplan-Meier curves showing disease-free survival and overall survival of Ccr2-knockout and wild-type LLC (top) and HNM007 (bottom) mice. Two-sided log-rank test. In FIGS. 2A-2F, two-sample, two-sided t-tests were used. Bars show mean ± s.e.m.

FIGS. 3A-3E. Low-dose AET skews monocytic MDSCs towards an interstitial macrophage-like population in the lung premetastatic microenvironment. FIG. 3A: GSEA analysis revealed that macrophage or myeloid differentiation and activation gene sets were upregulated (left) in monocytic MDSCs from the lungs of LLC mice treated with low-dose AET. Representative upregulated GSEA plots with core-enriched genes (middle and right). NES, normalized enrichment score. Color gradation is representative of log2-transformed fold change over mock (n=3 biological replicates). Gene-set enrichment P values, NES values and FDR values reported are calculated with 1,000 permutations in the GSEA software. FDR q < 0.25 was deemed significant. Gpr116 is also known as Adgrf5. FIG. 3B: Significant changes of representative transcription factors (FDR-adjusted P < 0.05) associated with monocytic differentiation (n=3 biological replicates). FIG. 3C: FACS showing the differentiation of sorted monocytic MDSCs in vitro. Splenic monocytic MDSCs from LLC mice were cultured for 3 days with tumor-conditioned medium (n=3 biological replicates). FIG. 3D: The top 200 significantly upregulated genes (FDR-adjusted P < 0.05) were mapped back to the reference ImmGen populations. The following populations from naive mice were used: lung CD103⁺ dendritic cells (ImmGen code, DC_103⁺11b-_Lu); CD11b⁺CD24⁺ lung dendritic cells (DC_103-11b⁺24⁺_Lu); lung interstitial macrophages (MF_11c-11b⁺_Lu); lung alveolar macrophages (MF_Alv_Lu); and lung monocytes (Mo Lu). The expressions of these 200 genes in 6 populations were transformed by zero-mean normalization. Two-sided Mann-Whitney U-test. n=3 biological replicates. FIG. 3E: FACS showing that low-dose AET can skew the differentiation of transferred monocytic MDSCs towards an interstitial macrophage-like phenotype in vivo. Purified 5×10⁶ CD45.1⁺ monocytic MDSCs from bone marrow (day 0) were adoptively transferred into CD45.2⁺ recipient mice within 24 h of resection (n=3 in each group) in the LLC model. Mice received low-dose AET or vehicle treatment for 36 h. In FIGS. 3B, 3C and 3E, two-sample, two-sided t-tests were used. All bars show mean ± s.e.m.

FIGS. 4A-4E. Low-dose AET inhibits pulmonary metastases and prolongs overall survival in mouse models, mainly by affecting MDSCs. FIG. 4A: Representative H &E-stained images of lung sections from LLC mice treated with low-dose AET or vehicle at different time points after surgery. Scale bars, 2 mm. Graph shows the area and numbers of metastatic nodules. At each time point, three mice were killed for analysis. For each sample, sections from three levels were analyzed. Tumor area was quantified using Aperio Imagescope software. Two-sample, two-sided t-test. All bars are mean ± s.e.m. FIG. 4B: Kaplan-Meier curves showing the disease-free survival and overall survival of LLC mice treated with low-dose AET or vehicle. FIG. 4C: Kaplan-Meier curves showing the disease-free survival and overall survival of LLC mice treated with paclitaxel plus cisplatin chemotherapy, low-dose AET or vehicle. FIG. 4D: Kaplan-Meier curves showing the disease-free survival and overall survival of mice treated with vehicle, IgG (isotype control), anti-CD4/CD8 (T-cell-depletion antibody), low-dose AET and anti-CD4/CD8 in combination with low-dose AET in the LLC model. FIG. 4E: Kaplan- Meier curves showing the disease-free survival and overall survival of mice treated with vehicle, CCR2 antagonist (RS504393) (Sigma), low-dose AET and RS504393 in combination with low-dose AET in the LLC model. In FIGS. 4B-4E, two-sided log-rank tests were used. *P < 0.05, **P < 0.01, ***P < 0.001.

FIGS. 5A-5C. Efficacy of low-dose AET on cancer recurrence in patients with stage I (T1-2aN0) NSCLC in a phase-II clinical trial. FIG. 5A: Schema for a randomized phase-II clinical trial of AET in patients with stage I (T1-2aN0) NSCLC (NCT01207726). FIG. 5B: Postsurgical recurrence rates in the observation and AET groups. FIG. 5C: Kaplan-Meier curves for disease-free survival in the observation and AET groups. P=0.50 by two-sided log-rank test.

FIGS. 6A-6E. Schema outlining the establishment and characteristics of the mouse models of pulmonary metastasis. FIG. 6A: Schema for establishing the highly aggressive HNM007 model. Pulmonary metastases were collected and serially subcutaneously implanted in the right flanks of mice for 10 passages. FIG. 6B: Schema for establishing the 4T1 model. FIG. 6C: Characteristics of mouse cell line (nonselective) and spontaneous mouse tissue models of pulmonary metastasis (metastases collected selectively from serial pulmonary metastases to produce a solely pulmonary-metastatic phenotype). FIG. 6D: Longitudinal gross pathological photographs of bilateral pulmonary metastases during the natural history of the LLC model in C57BL/6 mice from day 0 to day 15 after surgery. N1, N2 and N3 depict the experiment performed in triplicate. Two mice were killed at each time point from day 0 to day 15 (n=36); data for 18 mice are shown here as representative photomicrographs. FIG. 6E: H&E staining of pulmonary metastases in LLC, HNM007 and 4T1 mice. The histology of LLC (day 9), HNM007 (day 12) and 4T1 (day 12) pulmonary metastases were confirmed by a pathologist. Scale bars, 100 µm. Representative data were repeated at least three times with similar results.

FIGS. 7A-7C. CD11b⁺GR1⁺ cells persist as the predominant immune cells even after resection in the lung premetastatic microenvironment as functional MDSCs. FIG. 7A: In LLC mice, lung CD11b⁺Ly6C^{high}Ly6G⁻ and CD11b⁺Ly6C_{low}Ly6G⁺ cells collected at 72 h after resection both have suppressive activity in vitro against CD8a T cells. Freshly isolated CD11b⁺Ly6C^{high}Ly6G⁻ or CD11b⁺Ly6C_{low}Ly6G⁺ cells from both lungs at day 3 after resection were co-cultured with CD8a T cells for 72 h at different ratios (0:1, 1:1, 2:1, 4:1 and 1:0). T cell proliferation and IFNγ concentrations in the supernatant were measured by FACS (left) and ELISA (right), respectively (n=3 biological replicates). Representative data were repeated at least three times with similar results. Two-sample, two-sided t-test was used in the comparison with mock (CD8a T cells alone). FIG. 7B: Immune-cell profiles of liver in LLC mice. Single-cell suspensions from the entire liver were analyzed by FACS (n=3 mice per time point) at different time points after surgery. NC, negative control (normal liver from C57BL/6 mice). FIG. 7C: Immune-cell profiles of both lungs in HNM007 mice at different time points after surgery. Single-cell suspensions from both lungs were analyzed by FACS (n=3 mice per time point). NC, negative control (normal lungs from C57BL/6 mice). In FIGS. 7B, 7C, a two-sample, two-sided t-test was used in comparison with the negative control. All bars show mean ± s.e.m. *P < 0.05, **P < 0.01, ***P < 0.001.

FIGS. 8A-8H. Consideration of combined AET dosing on the basis of its effect on mouse models. FIG. 8A: Top, effect of different dosages of epigenetic modifiers on the viability of LLC1, HNM007 and 4T1 cells in vitro (72 h, Cell Counting Kit-8). Graphs show the mean of 3 independent experiments; two-sample, two-sided t-tests compared with mock. Bottom, effect of low-dose 5-azacytidine (100 nM) plus entinostat (50 nM) on the proliferation of LLC1, HNM007 and 4T1 cells in vitro. A total of 1×10⁵ viable cells was plated per well. Cells were collected at 24, 48 and 72 h and counted using a cell counter (Bio-Rad) after Trypan blue exclusion. Graphs show the mean of 3 independent experiments; significance at 72 h was determined by one-way ANOVA followed by Tukey’s test for multiple comparisons. FIG. 8B: The effect of low-dose 5-azacytidine (100 nM) plus entinostat (50 nM) on the viability of MDSCs from bone marrow (day 3) of LLC mice (top) and HNM007 mice (bottom) in vitro (Cell Counting Kit-8). Graphs show the mean of 3 independent experiments. FIG. 8C: The effect of low-dose 5-azacytidine (100 nM) plus entinostat (50 nM) on the apoptosis of MDSCs from bone marrow (day 3) of LLC and HNM007 mice in vitro. Cell apoptosis was measured by FACS at 48 h. The bottom right quadrant (annexin-V⁺/7-AAD⁻) and top right quadrant (annexin-V⁺/7-AAD⁺) represent early and late apoptotic cells, respectively. Graphs show the percentage of total apoptosis (early and late apoptosis) in mock and treatment groups (n=3 biological replicates). FIG. 8D: Tumor growth and body weight of NSG mice bearing LLC tissue that were treated with different doses of entinostat plus 5-azacytidine. Significance at day 12 (top) and day 14 (bottom) was determined by one-way ANOVA followed by Tukey’s test for multiple comparisons. FIG. 8E: Summary table of tumor growth, body weight and treatment-related death of NSG mice bearing LLC tissue. Regimens in red indicate dosages with no effect on tumor growth, weight loss or treatment-related death. FIG. 8F: Tumor growth and body weight of NSG mice bearing HNM007 tissue that were treated with 5-azacytidine at 0.5 mg kg⁻¹ d⁻¹ plus entinostat at 5 mg kg⁻¹ d⁻¹ or vehicle. Significance at day 14 was determined by two-sample, two-sided t-test. FIGS. 8G,8H: The effect of low-dose AET on the proliferation (FIG. 8G) and apoptosis (FIG. 8H) of donor-derived CD45.1+ MDSCs from bone marrow in CD45.2 LLC mice. Proliferation and apoptosis of immature (MHC-II⁻) and mature (MHC-11+) CD45.1+ cells were measured by FACS at 36 h after transfusion (day 2). Graphs in g show the percentage of Ki67+ cells (n=3 mice per group). Graphs in h show the percentage of total apoptosis (early and late apoptosis) in mock-treated and low-dose-AET groups (n=3 mice per group). In b, c, g, h, two-sample, two-sided t-tests were used. All bars show mean ± s.e.m. *P < 0.05.

FIGS. 9A-9F. Low-dose AET disrupts the lung premetastatic microenvironment, mainly by affecting MDSCs. FIG. 9A: The effect of low-dose AET (5-azacytidine 0.5 mg kg⁻¹ d⁻¹ plus entinostat 2.5 mg kg⁻¹ d⁻¹) on MDSCs from the lung at day 3 after resection in 4T1 mice (n=3 mice per group). FIGS. 9B, 9C: Immunofluorescence staining of CD4⁺ and CD8⁺ T cells (FIG. 9B) or GR1⁺ cells (FIG. 9C) from the lung premetastatic microenvironment (day 3) in LLC mice with or without low-dose AET. Negative control was normal lungs from tumor-free C57BL/6 mice. Immunofluorescence staining was performed using CD4 (green) and CD8 (red) antibodies, or GR1 (red) antibodies. Merged images contain DAPI staining for cell nuclei (blue). Original magnification 20×. Representative data were repeated at least three times with similar results. FIGS. 9D, 9E: The mRNA (FIG. 9D) and protein (FIG. 9E) levels of representative molecular factors known to promote premetastatic microenvironment formation from both lungs of normal mice, and mock-or low-dose-AET-treated LLC mice (day 3) were measured by quantitative PCR and western blot. Two-sample, two-sided t-test for quantitative PCR experiments (n=3 biological replicates). All the experiments were performed in triplicate and similar results were obtained. FIG. 9F: Top, graph showing the percentages of donorderived cell subsets (CD45.1⁺ MDSC cells) in the lungs of LLC mice or of sham-surgery mice (tumor-naive recipient mice) 36 h after surgery. Bottom, graph showing the percentages of donor-derived cell subsets (CD45.1⁺ MDSC cells) in the lungs of low-dose-AET-or vehicle-treated sham-surgery mice (tumor-naive recipient mice) 36 h after surgery. Purified 5×10⁶ MDSCs from bone marrow of CD45.1 mice bearing LLC tumors (day 0) were adoptively transferred into CD45.2 recipient mice in the sham-surgery tumor-naive model or LLC model (n=3 mice per group). In FIGS. 9A, 9F, two-sample, two-sided t-tests were used. All bars show mean ± s.e.m. *P < 0.05, **P < 0.01, ***P < 0.001.

FIGS. 10A-10H. Low-dose AET induces substantial changes in immune-cell chemotaxis and migration in MDSCs in LLC mice. FIG. 10A: Left, schema showing the effect of low-dose AET on monocytic or polymorphonuclear MDSCs transferred from CD45.1 to CD45.2 C57BL/6 mice in the LLC model. CD45.1⁺ cells (transferred polymorphonuclear MDSCs) were identified in the lungs of the recipient mice and analyzed by flow cytometry (right, n=3 mice per group). FIG. 10B: Left, schema showing trafficking ability of adoptively transferred monocytic or polymorphonuclear MDSCs from low-dose-AET-treated or untreated CD45.1 mice in the LLC model. CD45.1⁺ cells (transferred polymorphonuclear MDSCs) were identified in the lungs of the recipient CD45.2 mice and analyzed by flow cytometry at 18 h after transfer (right, n=3 mice per group). FIG. 10C: Left, top 10 upregulated gene sets from GSEA of lung monocytic MDSCs after 72 h of treatment with low-dose AET. Middle and right, representative upregulated GSEA plots of immune-cell chemotaxis and migration. Color gradation is representative of log2-transformed fold change over mock (n=3 biologically replicates). Gene-set enrichment P values, NES values and FDR values reported are calculated with 1,000 permutations in the GSEA software. FDR q < 0.25 was deemed significant. FIG. 10D: DAVID analyzes of significantly downregulated genes using the KEGG Gene Ontology in MDSCs from bone marrow of LLC mice, treated or untreated with low-dose AET. Top 20 downregulated pathways are presented (n=3 biologically replicates). Hypergeometric test (FDR-adjusted P < 0.05). FIG. 10E: Low-dose AET significantly decreases the nuclear activation of p52 and RELB (OD₄₅₀ nm (mock versus low-dose AET), p52: 0.95 ± 0.035 versus 0.721 + 0.011, P=0.0034; RELB, 0.251 ± 0.012 versus 0.1 ± 0.003, P=0.0002), but not p50 and p65 in monocytic MDSCs from bone marrow of LLC mice in vivo. Nuclear lysates were incubated with oligonucleotides containing the NF-κB-binding consensus sequence, and specific antibodies were used to detect the different subunits within the bound complexes (n=3 biological replicates). FIG. 10F: FACS shows the effect of 30 mg kg⁻¹ d⁻¹ and 75 mg kg⁻¹ d⁻¹ of BMS-345541 (a highly selective IKB kinase inhibitor) on CCR2 expression in monocytic MDSCs from bone marrow of LLC mice on day 3 after surgery. The experiments were performed in triplicate, and similar results were obtained. FIG. 10G: CXCR1 and CXCR2 expression of polymorphonuclear MDSCs collected on day 3 from the bone marrow or lung detected by quantitative PCR (top) and FACS (bottom) in LLC mice treated with vehicle or with 72 h of low-dose AET (n=3 biological replicates). FIG. 10H: Transwell migration assay of sorted polymorphonuclear MDSCs from bone marrow of low-dose-AET-(72 h) or vehicle-treated LLC mice, induced by CXCL1 (20 ng ml⁻¹ and 50 ng ml⁻¹) for 120 min (n=3 biological replicates). In FIGS. 10A, 10B, 10E, 10G, 10H, two-sample, two-sided t-tests were used. All bars show mean ± s.e.m.

FIGS. 11A-11C. Low-dose AET promotes the differentiation of monocytic MDSCs towards macrophages in the LLC model. FIG. 11A: DAVID analyzes of the significantly downregulated and upregulated genes using the KEGG Gene Ontology in LLC mice, treated or untreated with low-dose AET (n=3 biological replicates). Hypergeometric test (FDR-adjusted P < 0.05). FIGS. 11B, 11C: The mRNA (FIG. 11B) and protein (FIG. 11C) levels of representative transcription factors were measured by quantitative PCR and western blot, respectively. In vitro, splenic monocytic MDSCs from LLC mice were cultured for 3 days with tumor-conditioned medium. In vivo, monocytic MDSCs from both lungs of mock-(day 3) and low-dose-AET-treated LLC mice (day 3) were sorted for analysis. Representative data were repeated at least three times with similar results. In FIG. 11B, a two-sample two-sided t-test was used, n=3 biological replicates. All bars show mean ± s.e.m.

FIGS. 12A-12E. Low-dose AET promotes the differentiation of monocytic MDSCs towards an interstitial macrophage-like population in the lung premetastatic microenvironment. FIG. 12A: Gating strategy used to identify and analyze lung interstitial macrophages in the lung premetastatic microenvironment by FACS. FIG. 12B: The effect of low-dose AET on lung interstitial macrophages from LLC mice. The percentage and cell counts of interstitial macrophages from both lungs in mock-and low-dose-AET-treated mice were analyzed by FACS at day 3 after surgery (n=3 mice per group). Two-sample, two-sided t-test. All bars show mean ± s.e.m. FIG. 12C: Gating strategy used to identify and analyze CD45.1⁺ lung interstitial macrophages from the lungs of recipient CD45.2 mice after the transfusion of CD45.1⁺ monocytic MDSCs. FIG. 12D: Kaplan- Meier curves showing the disease-free survival and overall survival of Ccr2-knockout LLC mice after transfusion of wild-type monocytic MDSCs (5×10⁶), low-dose-AET-treated (in vivo) wild-type monocytic MDSCs (5×10⁶) or vehicle at day 1 and day 4, respectively. FIG. 12E: Kaplan-Meier curves showing the disease-free survival and overall survival of the Ccr2-knockout LLC mice after transfusion of wild-type polymorphonuclear MDSCs (5×10⁶), low-dose-AET-treated (in vivo) wild-type polymorphonuclear MDSCs (5×10⁶) or vehicle at day 1 and day 4, respectively. In FIGS. 12D, 12E, two-sided log-rank tests were used.

FIGS. 13A-13G. Low-dose AET inhibits pulmonary metastases and prolongs overall survival in mouse models. FIG. 13A: Representative photographs showing lungs treated with vehicle or low-dose AET in LLC (day 6) and HNM007 (day 10) mice. The red arrows indicate the metastases. FIG. 13B: Representative CBCT images of lung metastases on day 6 after resection in LLC mice treated with vehicle or low-dose AET. The red arrows indicate the metastases. FIG. 13C: Representative H&E-stained images of lung sections from HNM007 (top) and 4T1 (bottom) mice treated with low-dose AET or vehicle at different time points after surgery. Scale bars, 2 mm. Graph shows area and numbers of metastatic nodules. At each time point, three mice were killed for analysis. For each sample, sections from three levels were analyzed. Two-sample, two-sided t-test. FIG. 13D: Kaplan-Meier curves showing the disease-free survival and overall survival of HNM007 and 4T1 mice, treated with low-dose AET (for the 4T1 model, 5-azacytidine 0.5 mg kg⁻¹ d⁻¹ plus entinostat 2.5 mg kg⁻¹ d⁻¹) or vehicle after surgery. FIG. 13E: FACS showing the effect of T-cell-depleting antibodies on CD4⁺ and CD8⁺ T cells in the peripheral blood of LLC mice. n=3 mice per group. Two-sample, two-sided t-test. FIG. 13F: Kaplan-Meier curves showing the disease-free survival and overall survival of LLC mice treated with vehicle, CCR2 antagonist (RS102895) (Sigma), low-dose AET and RS102895 plus low-dose AET after surgery. FIG. 13G: Kaplan-Meier curves showing the disease-free survival and overall survival of HNM007 mice treated with vehicle, CCR2 antagonist (RS504393) (Sigma), low-dose AET and RS504393 in combination with low-dose AET. In FIGS. 13D, 13F, 13G, two-sided log-rank tests were used. Representative data in a, b were repeated at least three times with similar results. All bars show mean ± s.e.m. *P < 0.05, **P < 0.01, ***P < 0.001.

FIG. 14. Representation of the effect of low-dose adjuvant epigenetic modifiers on lung metastases in the tumor pulmonary metastasis models. Graphic model showing the inhibition of pulmonary metastases by low-dose AET via its effect on MDSCs. First, low-dose AET can inhibit the trafficking of monocytic and polymorphonuclear MDSCs from the bone marrow to the premetastatic microenvironment by downregulating the expression of CCR2 and CXCR2, respectively. Second, even if MDSCs migrate to the lung, low-dose AET can skew the differentiation of monocytic MDSCs towards an interstitial macrophage-like phenotype in the lung premetastatic microenvironment. Therefore, low-dose AET can disrupt the lung premetastatic microenvironment, ultimately inhibiting pulmonary metastases.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Cancer recurrence after complete surgical resection remains an unresolved clinical problem^{l-4}. Bone marrow-derived myeloid cells are key factors in forming the pre-metastatic microenvironment required for disseminating tumor cells to engraft distant sites⁵⁻⁷. Presently, there are no effective interventions aimed at preventing premetastatic microenvironment formation^{7,8}. Here, after surgical removal of a primary tumor, the present inventors found that adjuvant epigenetic therapy can disrupt the pre-metastatic microenvironment and inhibit cancer recurrence by its selective effect on myeloid derived suppressor cells (MDSCs). In murine models of post-surgery pulmonary metastases, MDSCs play a major role in pre-metastatic microenvironment formation after surgical resection of the primary tumor. Adjuvant epigenetic therapy, using low dose DNA methyltransferase and histone deacetylase inhibitors 5-azacytidine (AZA) and entinostat, disrupt the formation of pre-metastatic niches, by inhibiting the trafficking of MDSCs through the downregulation of CCR2 and CXCR2 and by promoting MDSCs differentiation into interstitial macrophages. Decreased accumulation of MDSCs in the premetastatic lung resulted in longer disease-free and overall survival compared to chemotherapy in mouse models. The present data demonstrated that even after complete surgical resection of a primary tumor, MDSCs can still contribute to the development of pulmonary premetastatic niches and settlement of residual tumor cells. A combination of low dose adjuvant epigenetic modifiers to disrupt this pre-metastatic microenvironment and inhibit pulmonary metastases may represent a novel adjuvant approach for cancer.

### I. Definitions

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present. The term “inhibitor” is synonymous with the term antagonist.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., a demethylating agent, HDAC inhibitor, CCR2 inhibitor, CXCR2 inhibitor and/or immunotherapy. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of inhibiting metastases in patients following resection.

As used herein, the term “effective,” means adequate to accomplish a desired, expected, or intended result. More particularly, an “effective amount” or a “therapeutically effective amount” is used interchangeably and refers to an amount of demethylating agent, HDAC inhibitor, CCR2 inhibitor, CXCR2 inhibitor and/or immunotherapy necessary to provide the desired “treatment” (defined herein) or therapeutic effect, e.g., an amount that is effective to prevent, alleviate, treat or ameliorate symptoms of a disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The term “combination” refers to two or more therapeutic agents to treat a condition or disorder described herein. Such combination of therapeutic agents may be in the form of a single pill, capsule, or intravenous solution. However, the term “combination” also encompasses the situation when the two or more therapeutic agents are in separate pills, capsules, syringes or intravenous solutions. Likewise, the term “combination therapy” refers to the administration of two or more therapeutic agents to treat a therapeutic condition or disorder described herein. Such administration encompasses co-administration of these therapeutic agents in a substantially simultaneous manner, such as in a single capsule having a fixed ratio of active ingredients or in multiple, or in separate containers (e.g., pills, capsules, etc.) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The terms “co-administration” and “in combination with” include the administration of two or more therapeutic agents simultaneously, concurrently or sequentially within no specific time limits unless otherwise indicated. In one embodiment, the agents are present in the cell or in the subject’s body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. It is understood that the embodiments described herein are applicable to other types of cancers.

The term “proliferative” disorder or disease refers to unwanted cell proliferation of one or more subset of cells in a multicellular organism resulting in harm (i.e., discomfort or decreased life expectancy) to the multicellular organism. For example, as used herein, proliferative disorder or disease includes neoplastic disorders and other proliferative disorders.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in vivo) to provide the compound. Prodrugs can typically be prepared using well-known methods, such as those described in 1 Burger’s Medicinal Chemistry and Drug Discovery, 172-178, 949-982 (Manfred E. Wolff ed., 5th ed. 1995), and Design of Prodrugs (H. Bundgaard ed., Elselvier, New York 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human or animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

As used herein, and unless otherwise specified, the terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### II. Demethylating Agents

DNA demethylating agents useful in the methods provided herein include, but are not limited to, 5-azacytidine (azacytidine; AZA), 5-azadeoxycytidine (decitabine; DAC), SGI-110 (guadecitabine), zebularine and procaine. In one embodiment, the DNA demethylating agent is 5-azacytidine. 5-azacitidine is 4-amino-1-β-D-ribofuranozyl-s-triazin-2(1H)-one, also known as VIDAZA®. Its empirical formula is C₈H₁2N₄O₅, the molecular weight is 244. 5-azacitidine is a white to off-white solid that is insoluble in acetone, ethanol and methyl ketone; slightly soluble in ethanol/water (50/50), propylene glycol and polyethylene glycol; sparingly soluble in water, water-saturated octanol, 5% dextrose in water, N-methyl-2-pyrrolidone, normal saline and 5% Tween 80 in water, and soluble in dimethylsulfoxide (DMSO).

In one embodiment, the methods provided herein comprise administration or co-administration of one or more DNA demethylating agents. In one embodiment, the DNA demethylating agents are cytidine analogs. A cytidine analog referred to herein is intended to encompass the free base of the cytidine analog, or a salt, solvate, hydrate, cocrystal, complex, prodrug, precursor, metabolite, and/or derivative thereof. In certain embodiments, a cytidine analog referred to herein encompasses the free base of the cytidine analog, or a salt, solvate, hydrate, cocrystal or complex thereof. In certain embodiments, a cytidine analog referred to herein encompasses the free base of the cytidine analog, or a pharmaceutically acceptable salt, solvate, or hydrate thereof.

In certain embodiments, the cytidine analog is 5-azacytidine (5-azacitidine). In certain embodiments, the cytidine analog is 5-aza-2′-deoxycytidine (decitabine). In certain embodiments, the cytidine analog is 5-azacytidine (5-azacitidine) or 5-aza-2′-deoxycytidine (decitabine). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacytidine (6-aza-CR); 5,6-dihydro-5-azacytidine (dH-aza-C R); N⁴-pentyloxy-carbonyl-5′-deoxy-5-fluorocytidine (Capecitabine); N⁴-octadecyl-cytarabine; or elaidic acid cytarabine. In certain embodiments, the cytidine analogs provided herein include any compound which is structurally related to cytidine or deoxycytidine and functionally mimics and/or antagonizes the action of cytidine or deoxycytidine.

In addition to AZA and Decitabine, other nucleoside analogs that target DNMTs can be used including, but not limited to, 6-Thioguanine (Thioguanine, 6tG) (2-amino-1,7-dihydro-6h-purine-6-thione); 5-Fluoro-2′-deoxycytidine (FdCyd), pseudocytidine (ΨICyd) (2-amino-5-β-D-ribofuranosylpyrimidin-4(1H)-one);5,6-Dihydro-5-azacytidine (DHAC); fazarbine (Ara-AC) (1-β-D-arabinofuranosyl-5-azacytosine); zebularine (Zeb) (1-(β-D-ribofuranosyl)-1,2 dihydropyrimidin-2-one); 2′-Deoxy-5,6-dihydro-5-azacytidine (DHDAC, KP-1212); 4′-Thio-2′-deoxycytidine (TdCyd); and 5-aza-4′-thio-2′-deoxycytidine (5-aza-TdCyd).

In other embodiments, prodrugs of AZA and decitabine can be used including, but not limited to, SGI-110, CP-4200 (5-azacytidine-5′-elaidate); RX-3117 (TV-1360, Fluorocyclopentenylcytosine); NPEOC-DAC (2′-deoxy-N4-[2-(4-nitrophenyl)ethoxycarbonyl]-5-azacytidine); an d2′3′5′Triacetyl-5-azacytidine (TAC).

Certain embodiments herein provide salts, cocrystals, solvates (e.g., hydrates), complexes, prodrugs, precursors, metabolites, and/or other derivatives of the cytidine analogs provided herein. For example, particular embodiments provide salts, cocrystals, solvates (e.g., hydrates), complexes, precursors, metabolites, and/or other derivatives of 5-azacytidine. Certain embodiments herein provide salts, cocrystals, and/or solvates (e.g., hydrates) of the cytidine analogs provided herein. Certain embodiments herein provide salts and/or solvates (e.g., hydrates) of the cytidine analogs provided herein. Certain embodiments provide cytidine analogs that are not salts, cocrystals, solvates (e.g., hydrates), or complexes of the cytidine analogs provided herein. For example, particular embodiments provide 5-azacytidine in a non-ionized, non-solvated (e.g., anhydrous), non-complexed form. Certain embodiments herein provide a mixture of two or more cytidine analogs provided herein.

In one embodiment, the compound used in the methods provided herein is a free base, or a pharmaceutically acceptable salt or solvate thereof. In one embodiment, the free base or the pharmaceutically acceptable salt or solvate is a solid. In another embodiment, the free base or the pharmaceutically acceptable salt or solvate is a solid in an amorphous form. In yet another embodiment, the free base or the pharmaceutically acceptable salt or solvate is a solid in a crystalline form. For example, particular embodiments provide 5-azacytidine in solid forms, which can be prepared, for example, according to the methods described in U.S. Pat. Nos. 6,943,249, 6,887,855 and 7,078,518, and U.S. Pat. Application Publication Nos. 2005/027675 and 2006/247189, each of which is incorporated by reference herein in their entireties. In other embodiments, 5-azacytidine in solid forms can be prepared using other methods known in the art.

In one embodiment, the compound used in the methods provided herein is a pharmaceutically acceptable salt of the cytidine analog, which includes, but is not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, or undecanoate salts.

Cytidine analogs provided herein may be prepared using synthetic methods and procedures referenced herein or otherwise available in the literature. For example, particular methods for synthesizing 5-azacytidine are disclosed, e.g., in U.S. Pat. No. 7,038,038 and references discussed therein, each of which is incorporated herein by reference. Other cytidine analogs provided herein may be prepared, e.g., using procedures known in the art, or may be purchased from a commercial source. In one embodiment, the cytidine analogs provided herein may be prepared in a particular solid form (e.g., amorphous or crystalline form). See, e.g., U.S. Pat. Application Ser. No. 10/390,578, filed Mar. 17, 2003 and U.S. Pat. Application Ser. No. 10/390,530, filed Mar. 17, 2003, both of which are incorporated herein by reference in their entireties. In other embodiments, methods of synthesis include methods as disclosed in U.S. Pat. No. 7,038,038; U.S. Pat. No. 6,887,855; U.S. Pat. No. 7,078,518; U.S. Pat. No. 6,943,249; and U.S. Ser. No. 10/823,394, all incorporated by reference herein in their entireties.

### III. Histone Deacetylase (HDAC) Inhibitors

Histone deacetylases (HDAC) are enzymes capable of removing the acetyl group bound to the lysine residues in the N-terminal portion of histones or in other proteins. HDACs can be subdivided into four classes, on the basis of structural homologies. Class I HDACs (HDAC 1, 2, 3 and 8) are similar to the RPD3 yeast protein and are located in the cell nucleus. Class II HDACs (HDAC 4, 5, 6, 7, 9 and 10) are similar to the HDA1 yeast protein and arc located both in the nucleus and in the cytoplasm. Class III HDACs are a structurally distinct form of NAD-dependent enzymes correlated with the SIR2 yeast protein. Class IV (HDAC 11) consists at the moment of a single enzyme having particular structural characteristics. The HDACs of classes I, II and IV are zinc enzymes and can be inhibited by various classes of molecule: hydroxamic acid derivatives, cyclic tetrapeptides, short-chain fatty acids, aminobenzamides, derivatives of electrophilic ketones, and the like. Class III HDACs are not inhibited by hydroxamic acids, and their inhibitors have structural characteristics different from those of the other classes.

The expression “histone deacetylase inhibitor” in relation to the present invention is to be understood as meaning any molecule of natural, recombinant or synthetic origin capable of inhibiting the activity of at least one of the enzymes classified as HDAC. In particular embodiments, an HDAC inhibitor inhibits enzymes of Class I and II.

Examples of HDAC inhibitors useful in the compositions and methods of the present invention include, but are not limited to, givinostat, entinostat, trichostatin A (TSA), Vorinostat (SAHA), Valproic Acid (VPA), romidepsin (FK228, depsipeptide) and MS-275. In a specific embodiment, the HDAC inhibitor is givinostat (ITF2357; diethyl- [6-(4-hydroxycarbamoyl-phenylcarbamoyloxymethyl)-naphthalen-2-yl methyl] -ammonium chloride). See, e.g., WO97/43251 (anhydrous form) and in WO2004/065355 (monohydrate crystal form).

HDAC inhibitors also include tucidinostat (chidamide), panobinostat (Farydak, LBH589), belinostat (PXD101), mocetinostat (MGCD0103), abexinostat (PCI-24781), SB939, resminostat (4SC-201),quisinostat (JNJ26481585), Kevetrin, CUDC-101, AR-42, CHR-2845, CHR-3996, Domatinostat (4SC-202), ricolinostat (ACY-1215), and ME-344.

Examples of HDAC inhibitors also include ACY-738, BG45, Biphenyl-4-sulfonyl chloride, BRD73954, CAY10603, Citarinostat (ACY-241), CUDC-907, Dacinostat (LAQ824), Droxinostat, ITSA-1 (ITSA1), LMK-235, M344, MC1568, Nexturastat A, PCI-34051, Pracinostat (SB939), RG2833 (RGFP109), RGFP966, Santacruzamate A (CAY10683), SKLB-23bb, Sodium butyrate, Splitomicin, Suberohydroxamic acid, Tacedinaline (CI994), Tasquinimod, TH34, Tinostamustine (EDO-S101), TMP195, TMP269, Tubacin, Tubastatin A, Tubastatin A HCl, UF010, and WT161.

Other examples of HDAC inhibitors include those described in the following patent applications: WO2004/092115, WO2005/019174, WO2003/076422, WO1997/043251, WO2006/010750, WO2006/003068, WO2002/030879, WO2002/022577, WO1993/007148, WO2008/033747, WO2004/069823, EP0847992 and WO2004/071400, the contents of which are incorporated herein by reference in their entirety.

### IV. CCR2 Inhibitors/Antagonists

In some embodiments, a CCR2 inhibitor comprises CCX140 or CCX872 (ChemoCentryx, Inc. (Mountain View, CA)). In other embodiments, a CCR2 inhibitor comprises PF-04136309 (PF-6309) ((S)-N-(2-(3-((4-hydroxy-4-(5-(pyrimidin-2-yl)pyridin-2-yl)cyclohexyl)amino)pyrrolidin-1-yl)-2-oxoethyl)-3-(trifluoromethyl)benzamide) (Pfizer (New York, NY)).

In other embodiments, a CCR2 inhibitor comprises PF-04178903; INCB-8696; CCX-915; CCX-872; MLN-1202; CCX-140; PF-4136309; JNJ-17166864; AZD-2423; INCB-003284; BMS-741672; MK-0812; PF-04634817; CNTO888. In a specific embodiment, a CCR2 inhibitor comprises 747 (kaempferol 3-(2,4-di-E-p-coumaroylrhamnoside) (Yao et al., 22 EBIOMEDICINE 58-67 (2017)).

Other examples of CCR2 inhibitors include, but are not limited to, BMS CCR2 22 (2-[(Isopropylaminocarbonyl)amino]-N-[2-[[cis-2-[[4-(methylthio)benzoyl] amino] cyclohexyl] amino]-2-oxoethyl] -5-(trifluoromethyl)benzamide); INCB 3284 dimesylate (N-[2-[[(3R)-1-[trans-4-Hydroxy-4-(6-methoxy-3-pyridinyl)cyclohexyl]-3-pyrrolidinyl]amino]-2-oxoethyl]-3-(trifluoromethyl)-benzamide dimethanesulfonate); JNJ 27141491 (3-[(1S)-1-(3,4-Difluorophenyl)propyl]-2,3-dihydro-5-(5-isoxazolyl)-2-thioxo-1H-imidazole-4-carboxylic acid methyl ester); RS 102895 hydrochloride (1′-[2-[4-(Trifluoromethyl)phenyl]ethyl]-spiro[4H-3,1-benzoxazine-4,4′-piperidin]-2(1H)-one hydrochloride); RS 504393 (6-Methyl-1′-[2-(5-methyl-2-phenyl-4-oxazolyl)ethyl]-spiro[4H-3,1-benzoxazine-4,4′-piperidin]-2(1H)-one); and Teijin compound 1 (N-[2-[[(3R)-1-[(4-chlorophenyl)methyl]-3-pyrrolidinyl]amino]-2-oxoethyl]-3-(trifluoromethyl)benzamide hydrochloride).

Example of protein/peptide CCR2 inhibitors include anti-CCR2 mAb LS-132.1D9; and MCP-1 (9-76), also referred to as MCP-1 7-ND (Gong et al., 186(1) J. EXP. MED. 131-37 (1997)).

### V. CXCR2 Inhibitors/Antagonists

In certain embodiments, a CXCR2 inhibitor comprises AZD5069 ([N-(2-(2,3-difluorobenzylthio)-6-((2R,3S)-3,4-dihydroxybutan-2-yloxy)[2,4,5,6-¹³C4, 1,3-¹⁵N2]pyrimidin-4-yl)azetidine-1-sulfonamide,[¹⁵N2,¹³C4]N-(2-(2,3-difluoro-6-[3H]-benzylthio)-6-((2R,3S)-3,4-dihydroxybutan-2-yloxy)pyrimidin-4-yl)azetidine-1-sulfonamide]) (AstraZeneca (Wilmington, DE)). In other embodiments, a CXCR2 inhibitor comprises MK-7123 (SCH527123, Navarixin) (2-hydroxy-N,N-dimethyl-3-[[2-[[(1R)-1-(5-methylfuran-2-yl)propyl]amino]-3,4-dioxocyclobuten-1-yl]amino]benzamide) (Merck & Co. (Kenilworth, NJ)).

In particular embodiments, a CXCR2 inhibitor includes, but is not limited to, SB-332235; danirixin; elubrixin; PS-291822; SB225002; SX-682; SX-576; SX-517; ladarixin (PubChem CID 11372270; meraxin, DF2156A); reparixin (PubChem CID 9838712; repertaxin, DF1681B); reparixin L-lysine salt (PubChem CID 9932389); DF2755A, CXCL8 fragment comprising amino acids 3-74 and substitutions K11R/G31P (G31P); DF2162 (PubChem CID 11289471); and SCH-479833.

### VI. Pharmaceutical Compositions and Formulations

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the demethylating agent, HDAC inhibitor, CCR2 inhibitor and/or CXCR2 inhibitor are administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a demethylating agent and an HDAC inhibitor, and a CCR2 inhibitor and/or a CXCR2 inhibitor together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection.

Optimal precision in achieving concentrations of the therapeutic regimen (e.g., pharmaceutical compositions comprising a demethylating agent and an HDAC inhibitor in combination with another therapeutic agent such as a CCR2 and/or CXCR2 inhibitor) within the range that yields maximum efficacy with minimal toxicity may require a regimen based on the kinetics of the pharmaceutical composition’s availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

### VII. Methods of Treatment

In one embodiment, an effective amount of an epigenetic therapy (e.g., a demethylating agent and/or an HDAC inhibitor), as well as a CCR2 inhibitor and/or a CXCR2 inhibitor to be used is a therapeutically effective amount. In one embodiment, the amounts of the drugs to be used in the methods provided herein include an amount sufficient to cause improvement in at least a subset of patients with respect to symptoms, overall course of disease, or other parameters known in the art. Precise amounts for therapeutically effective amounts in the pharmaceutical compositions and methods will vary depending on the age, weight, disease, and condition of the patient, as well as the particular drug being administered.

In particular embodiments, the demethylating agent is administered by, e.g., intravenous (IV), subcutaneous (SC) or oral routes. Certain embodiments herein provide co-administration of the demethylating agent with one or more additional active agents to provide a synergistic therapeutic effect in subjects in need thereof. The co-administered agent(s) may be a cancer therapeutic agent, as described herein. In particular embodiments, the co-administered agents comprise an HDAC inhibitor, a CCR2 inhibitor and/or a CXCR2 inhibitor. In certain embodiments, the co-administered agent(s) may be dosed, e.g., orally or by injection (e.g., IV or SC).

Certain embodiments herein provide methods for inhibiting cancer metastases comprising administering the demethylating agent using, e.g., IV, SC and/or oral administration methods. In certain embodiments, treatment cycles comprise multiple doses administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. For example, in certain embodiments, the amount of the demethylating agent administered in the methods provided herein may range, e.g., between about 30 mg/m²/day and about 2,000 mg/m²/day, between about 100 mg/m²/day and about 1,000 mg/m²/day, between about 100 mg/m²/day and about 500 mg/m²/day, between about 30 mg/m²/day and about 500 mg/m²/day, between about 30 mg/m²/day and about 200 mg/m²/day, between about 30 mg/m²/day and about 100 mg/m²/day, between about 30 mg/m²/day and about 75 mg/m²/day, or between about 120 mg/m²/day and about 250 mg/m²/day. In certain embodiments, particular dosages are, e.g., about 30 mg/m²/day, about 40 mg/m²/day, about 50 mg/m²/day, about 60 mg/m²/day, about 75 mg/m²/day, about 80 mg/m²/day, about 100 mg/m²/day, about 120 mg/m²/day, about 140 mg/m²/day, about 150 mg/m²/day, about 180 mg/m²/day, about 200 mg/m²/day, about 220 mg/m²/day, about 240 mg/m²/day, about 250 mg/m²/day, about 260 mg/m²/day, about 280 mg/m²/day, about 300 mg/m²/day, about 320 mg/m²/day, about 350 mg/m²/day, about 380 mg/m²/day, about 400 mg/m²/day, about 450 mg/m²/day, or about 500 mg/m²/day. In certain embodiments, particular dosages are, e.g., up to about 30 mg/m²/day, up to about 40 mg/m²/day, up to about 50 mg/m²/day, up to about 60 mg/m²/day, up to about 70 mg/m²/day, up to about 80 mg/m²/day, up to about 90 mg/m²/day, up to about 100 mg/m²/day, up to about 120 mg/m²/day, up to about 140 mg/m²/day, up to about 150 mg/m²/day, up to about 180 mg/m²/day, up to about 200 mg/m²/day, up to about 220 mg/m²/day, up to about 240 mg/m²/day, up to about 250 mg/m²/day, up to about 260 mg/m²/day, up to about 280 mg/m²/day, up to about 300 mg/m²/day, up to about 320 mg/m²/day, up to about 350 mg/m²/day, up to about 380 mg/m²/day, up to about 400 mg/m²/day, up to about 450 mg/m²/day, up to about 500 mg/m²/day, up to about 750 mg/m²/day, or up to about 1000 mg/m²/day. In a specific non-limiting embodiment, the dose of the demethylating agent is about 40 mg/m².

In one embodiment, the amount of the demethylating agent administered in the methods provided herein may range, e.g., between about 5 mg/day and about 2,000 mg/day, between about 10 mg/day and about 2,000 mg/day, between about 20 mg/day and about 2,000 mg/day, between about 50 mg/day and about 1,000 mg/day, between about 100 mg/day and about 1,000 mg/day, between about 100 mg/day and about 500 mg/day, between about 150 mg/day and about 500 mg/day, or between about 150 mg/day and about 250 mg/day. In certain embodiments, particular dosages are, e.g., about 10 mg/day, about 20 mg/day, about 50 mg/day, about 75 mg/day, about 100 mg/day, about 120 mg/day, about 150 mg/day, about 200 mg/day, about 250 mg/day, about 300 mg/day, about 350 mg/day, about 400 mg/day, about 450 mg/day, about 500 mg/day, about 600 mg/day, about 700 mg/day, about 800 mg/day, about 900 mg/day, about 1,000 mg/day, about 1,200 mg/day, or about 1,500 mg/day. In certain embodiments, particular dosages are, e.g., up to about 10 mg/day, up to about 20 mg/day, up to about 50 mg/day, up to about 75 mg/day, up to about 100 mg/day, up to about 120 mg/day, up to about 150 mg/day, up to about 200 mg/day, up to about 250 mg/day, up to about 300 mg/day, up to about 350 mg/day, up to about 400 mg/day, up to about 450 mg/day, up to about 500 mg/day, up to about 600 mg/day, up to about 700 mg/day, up to about 800 mg/day, up to about 900 mg/day, up to about 1,000 mg/day, up to about 1,200 mg/day, or up to about 1,500 mg/day.

In one embodiment, the amount of the demethylating agent in the pharmaceutical composition or dosage form provided herein may range, e.g., between about 5 mg and about 2,000 mg, between about 10 mg and about 2,000 mg, between about 20 mg and about 2,000 mg, between about 30 mg and about 1,000 mg, between about 30 mg and about 500 mg, between about 30 mg and about 250 mg, between about 100 mg and about 500 mg, between about 150 mg and about 500 mg, or between about 150 mg and about 250 mg. In certain embodiments, particular amounts are, e.g., about 10 mg, about 20 mg, about 30 mg, about 40 mg, about 50 mg, about 75 mg, about 100 mg, about 120 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1,000 mg, about 1,200 mg, or about 1,500 mg. In certain embodiments, particular amounts are, e.g., up to about 10 mg, up to about 20 mg, up to about 30 mg, up to about 40 mg, up to about 50 mg, up to about 75 mg, up to about 100 mg, up to about 120 mg, up to about 150 mg, up to about 200 mg, up to about 250 mg, up to about 300 mg, up to about 350 mg, up to about 400 mg, up to about 450 mg, up to about 500 mg, up to about 600 mg, up to about 700 mg, up to about 800 mg, up to about 900 mg, up to about 1,000 mg, up to about 1,200 mg, or up to about 1,500 mg.

In one embodiment, depending on the disease to be treated and the subject’s condition, the demethylating agent may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The demethylating agent may be formulated, alone or together with one or more active agent(s), in suitable dosage unit with pharmaceutically acceptable excipients, carriers, adjuvants and vehicles, appropriate for each route of administration. In one embodiment, the demethylating agent is administered orally. In another embodiment, the demethylating agent is administered parenterally. In yet another embodiment, the demethylating agent is administered intravenously.

In one embodiment, the demethylating agent can be delivered as a single dose such as, e.g., a single bolus injection, or oral tablets or pills; or over time such as, e.g., continuous infusion over time or divided bolus doses over time. In one embodiment, the demethylating agent can be administered repetitively if necessary, for example, until the patient experiences stable disease or regression, or until the patient experiences disease progression or unacceptable toxicity.

In one embodiment, the demethylating agent can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, the demethylating agent is administered daily, for example, once or more than once each day for a period of time. In one embodiment, the demethylating agent is administered daily for an uninterrupted period of at least 7 days, in some embodiments, up to 52 weeks. In one embodiment, the demethylating agent is administered intermittently, i.e., stopping and starting at either regular or irregular intervals. In one embodiment, the demethylating agent is administered for one to six days per week. In one embodiment, the demethylating agent is administered in cycles (e.g., daily administration for two to eight consecutive weeks, then a rest period with no administration for up to one week; or e.g., daily administration for one week, then a rest period with no administration for up to three weeks). In one embodiment, the demethylating agent is administered on alternate days. In one embodiment, the demethylating agent is administered in cycles (e.g., administered daily or continuously for a certain period interrupted with a rest period).

In one embodiment, the frequency of administration ranges from about daily to about monthly. In certain embodiments, the demethylating agent is administered once a day, twice a day, three times a day, four times a day, once every other day, twice a week, once every week, once every two weeks, once every three weeks, or once every four weeks. In one embodiment, the demethylating agent is administered once a day. In another embodiment, the demethylating agent is administered twice a day. In yet another embodiment, the demethylating agent is administered three times a day. In still another embodiment, the demethylating agent is administered four times a day.

In one embodiment, the demethylating agent is administered once per day from one day to six months, from one week to three months, from one week to four weeks, from one week to three weeks, or from one week to two weeks. In certain embodiments, the demethylating agent is administered once per day for one week, two weeks, three weeks, or four weeks. In one embodiment, the demethylating agent is administered once per day for one week. In another embodiment, the demethylating agent is administered once per day for two weeks. In yet another embodiment, the demethylating agent is administered once per day for three weeks. In still another embodiment, the demethylating agent is administered once per day for four weeks.

In one embodiment, the demethylating agent is administered once per day for about 1 week, about 2 weeks, about 3 weeks, about 4 weeks, about 6 weeks, about 9 weeks, about 12 weeks, about 15 weeks, about 18 weeks, about 21 weeks, or about 26 weeks. In certain embodiments, the demethylating agent is administered intermittently. In certain embodiments, the demethylating agent is administered intermittently in the amount of between about 30 mg/m²/day and about 2,000 mg/m²/day. In certain embodiments, the demethylating agent is administered continuously. In certain embodiments, the demethylating agent is administered continuously in the amount of between about 30 mg/m²/day and about 1,000 mg/m²/day.

In certain embodiments, the demethylating agent is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, the demethylating agent is administered to a patient in cycles. In one embodiment, a method provided herein comprises administering the demethylating agent in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or greater than about 30 cycles.

In one embodiment, the demethylating agent is administered to a patient at a dose provided herein over a cycle of 28 days which consists of a 7-day treatment period and a 21-day resting period. In one embodiment, the demethylating agent is administered to a patient at a dose provided herein each day from day 1 to day 7, followed with a resting period from day 8 to day 28 with no administration of the demethylating agent. In one embodiment, the demethylating agent is administered to a patient in cycles, each cycle consisting of a 7-day treatment period followed with a 21-day resting period. In particular embodiments, the demethylating agent is administered to a patient at a dose of about 50, about 60, about 70, about 75, about 80, about 90, or about 100 mg/m²/d, for 7 days, followed with a resting period of 21 days. In one embodiment, the demethylating agent is administered intravenously. In one embodiment, the demethylating agent is administered subcutaneously. In other embodiments, the demethylating agent is administered orally in cycles.

Accordingly, in one embodiment, the demethylating agent is administered daily in single or divided doses for about one week, about two weeks, about three weeks, about four weeks, about five weeks, about six weeks, about eight weeks, about ten weeks, about fifteen weeks, or about twenty weeks, followed by a rest period of about 1 day to about ten weeks. In one embodiment, the methods provided herein contemplate cycling treatments of about one week, about two weeks, about three weeks, about four weeks, about five weeks, about six weeks, about eight weeks, about ten weeks, about fifteen weeks, or about twenty weeks. In some embodiments, the demethylating agent is administered daily in single or divided doses for about one week, about two weeks, about three weeks, about four weeks, about five weeks, or about six weeks with a rest period of about 1, 3, 5, 7, 9, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, the methods provided herein comprise: i) administering to the subject a first daily dose of the demethylating agent; ii) optionally resting for a period of at least one day where the demethylating agent is not administered to the subject; iii) administering a second dose of the demethylating agent to the subject; and iv) repeating steps ii) to iii) a plurality of times. In certain embodiments, the first daily dose is between about 30 mg/m²/day and about 2,000 mg/m²/day. In certain embodiments, the second daily dose is between about 30 mg/m²/day and about 2,000 mg/m²/day. In certain embodiments, the first daily dose is higher than the second daily dose. In certain embodiments, the second daily dose is higher than the first daily dose. In one embodiment, the rest period is 2 days, 3 days, 5 days, 7 days, 10 days, 12 days, 13 days, 14 days, 15 days, 17 days, 21 days, or 28 days. In one embodiment, the rest period is at least 2 days and steps ii) through iii) are repeated at least three times. In one embodiment, the rest period is at least 2 days and steps ii) through iii) are repeated at least five times. In one embodiment, the rest period is at least 3 days and steps ii) through iii) are repeated at least three times. In one embodiment, the rest period is at least 3 days and steps ii) through iii) are repeated at least five times. In one embodiment, the rest period is at least 7 days and steps ii) through iii) are repeated at least three times. In one embodiment, the rest period is at least 7 days and steps ii) through iii) are repeated at least five times. In one embodiment, the rest period is at least 14 days and steps ii) through iii) are repeated at least three times. In one embodiment, the rest period is at least 14 days and steps ii) through iii) are repeated at least five times. In one embodiment, the rest period is at least 21 days and steps ii) through iii) are repeated at least three times. In one embodiment, the rest period is at least 21 days and steps ii) through iii) are repeated at least five times. In one embodiment, the rest period is at least 28 days and steps ii) through iii) are repeated at least three times. In one embodiment, the rest period is at least 28 days and steps ii) through iii) are repeated at least five times. In one embodiment, the methods provided herein comprise: i) administering to the subject a first daily dose of the demethylating agent for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; iii) administering to the subject a second daily dose of the demethylating agent for 1, 2, 3, 4, 5, 6, 7, 8, 9, 10, 11, 12, 13, or 14 days; and iv) repeating steps ii) to iii) a plurality of times. In one embodiment, the methods provided herein comprise: i) administering to the subject a daily dose of the demethylating agent for 1, 2, 3, 4, 5, 6, 7, 8, 9, 10, 11, 12, 13, or 14 days; ii) resting for a period of 1, 2, 3, 4, 5, 6, 7, 8, 9, 10, 11, 12, 13, 14, 15, 16, 17, 18, 19, 20, 21, 22, 23, 24, 25, 26, 27, or 28 days; and iii) repeating steps i) to ii) a plurality of times. In one embodiment, the methods provided herein comprise: i) administering to the subject a daily dose of the demethylating agent for 7 days; ii) resting for a period of 21 days; and iii) repeating steps i) to ii) a plurality of times. In one embodiment, the daily dose is between about 30 mg/m²/day and about 2,000 mg/m²/day. In one embodiment, the daily dose is between about 30 mg/m²/day and about 1,000 mg/m²/day. In one embodiment, the daily dose is between about 30 mg/m²/day and about 500 mg/m²/day. In one embodiment, the daily dose is between about 30 mg/m²/day and about 200 mg/m²/day. In one embodiment, the daily dose is between about 30 mg/m²/day and about 100 mg/m²/day.

In certain embodiments, the demethylating agent is administered continuously for between about 1 and about 52 weeks. In certain embodiments, the demethylating agent is administered continuously for about 1, 2, 3, 4, 5, 6, 7, 8, 9, 10, 11, or 12 months. In certain embodiments, the demethylating agent is administered continuously for about 14, about 28, about 42, about 84, or about 112 days. It is understood that the duration of the treatment may vary with the age, weight, and condition of the subject being treated, and may be determined empirically using known testing protocols or according to the professional judgment of the person providing or supervising the treatment. The skilled clinician will be able to readily determine, without undue experimentation, an effective drug dose and treatment duration, for treating an individual subject having a particular type of cancer.

In one embodiment, pharmaceutical compositions may contain sufficient quantities of the demethylating agent to provide a daily dosage of about 10 to 150 mg/m² (based on patient body surface area) or about 0.1 to 4 mg/kg (based on patient body weight) as single or divided (2-3) daily doses. In one embodiment, dosage is provided via a seven-day administration of 75 mg/m² subcutaneously, once every twenty-eight days, for as long as clinically necessary. In one embodiment, dosage is provided via a seven-day administration of 100 mg/m² subcutaneously, once every twenty-eight days, for as long as clinically necessary. In one embodiment, up to 4, up to 5, up to 6, up to 7, up to 8, up to 9 or more 28-day cycles are administered. Other methods for providing an effective amount of the demethylating agent are disclosed in, for example, “Colon-Targeted Oral Formulations of Cytidine Analogs”, U.S. Ser. No. 11/849,958, and “Oral Formulations of Cytidine Analogs and Methods of Use Thereof”, U.S. Ser. No. 12/466,213, both of which are incorporated by reference herein in their entireties.

In particular embodiments, the number of cycles administered is, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 22, at least 24, at least 26, at least 28, at least 30, at least 32, at least 34, at least 36, at least 38, at least 40, at least 42, at least 44, at least 46, at least 48, or at least 50 cycles of the demethylating agent treatment. In particular embodiments, the treatment is administered, e.g., 1, 2, 3, 4, 5, 6, 7, 8, 9, 10, 11, 12, 13, or 14 days out of a 28-day period. In particular embodiments, the demethylating agent dose is, e.g., at least 10 mg/day, at least 20 mg/day, at least 30 mg/day, at least 40 mg/day, at least 50 mg/day, at least 55 mg/day, at least 60 mg/day, at least 65 mg/day, at least 70 mg/day, at least 75 mg/day, at least 80 mg/day, at least 85 mg/day, at least 90 mg/day, at least 95 mg/day, or at least 100 mg/day.

In particular embodiments, the dosing is performed, e.g., subcutaneously or intravenously. In particular embodiments, the contemplated specific the demethylating agent dose is, e.g., at least 30 mg/m²/day, at least 40 mg/m²/day, at least 50 mg/m²/day, at least 60 mg/m²/day, at least 70 mg/m²/day, at least 75 mg/m²/day, at least 80 mg/m2/day, at least 90 mg/m²/day, or at least 100 mg/m²/day. One particular embodiment herein provides administering the treatment for 7 days out of each 28-day period. One particular embodiment herein provides a dosing regimen of 75 mg/m² subcutaneously or intravenously, daily for 7 days. One particular embodiment herein provides a dosing regimen of 100 mg/m² subcutaneously or intravenously, daily for 7 days.

In one embodiment, an HDAC inhibitor (e.g., givinostat, entinostat, romidepsin (FK228, depsipeptide) and the like) is administered intravenously. In one embodiment, the HDAC inhibitor is administered intravenously over a 1-6 hour period. In one embodiment, the HDAC inhibitor is administered intravenously over a 3-4 hour period. In one embodiment, the HDAC inhibitor is administered intravenously over a 5-6 hour period. In one embodiment, the HDAC inhibitor is administered intravenously over a 4 hour period.

In one embodiment, the HDAC inhibitor is administered in a dose ranging from 0.5 mg/m² to 28 mg/m². In one embodiment, the HDAC inhibitor is administered in a dose ranging from 0.5 mg/m² to 5 mg/m². In one embodiment, the HDAC inhibitor is administered in a dose ranging from 1 mg/m² to 25 mg/m². In one embodiment, the HDAC inhibitor is administered in a dose ranging from 1 mg/m² to 20 mg/m². In one embodiment, the HDAC inhibitor is administered in a dose ranging from 1 mg/m² to 15 mg/m². In one embodiment, the HDAC inhibitor is administered in a dose ranging from 2 mg/m² to 15 mg/m². In one embodiment, the HDAC inhibitor is administered in a dose ranging from 2 mg/m² to 12 mg/m². In one embodiment, the HDAC inhibitor is administered in a dose ranging from 4 mg/m² to 12 mg/m². In one embodiment, the HDAC inhibitor is administered in a dose ranging from 6 mg/m² to 12 mg/m². In one embodiment, the HDAC inhibitor is administered in a dose ranging from 8 mg/m² to 12 mg/m². In one embodiment, the HDAC inhibitor is administered in a dose ranging from 8 mg/m² to 10 mg/m². In one embodiment, the HDAC inhibitor is administered in a dose of about 8 mg/m². In one embodiment, the HDAC inhibitor is administered in a dose of about 9 mg/m². In one embodiment, the HDAC inhibitor is administered in a dose of about 10 mg/m². In one embodiment, the HDAC inhibitor is administered in a dose of about 11 mg/m². In one embodiment, the HDAC inhibitor is administered in a dose of about 12 mg/m². In one embodiment, the HDAC inhibitor is administered in a dose of about 13 mg/m². In one embodiment, the HDAC inhibitor is administered in a dose of about 14 mg/m². In one embodiment, the HDAC inhibitor is administered in a dose of about 15 mg/m².

In one embodiment, the HDAC inhibitor is administered in a dose of 14 mg/m² over a 4 hour iv infusion on days 1, 8 and 15 of the 28 day cycle. In one embodiment, the cycle is repeated every 28 days.

In one embodiment, increasing doses of the HDAC inhibitor are administered over the course of a cycle. In one embodiment, the dose of about 8 mg/m² followed by a dose of about 10 mg/m², followed by a dose of about 12 mg/m² is administered over a cycle.

In one embodiment, the HDAC inhibitor is administered orally. In one embodiment, the HDAC inhibitor is administered in a dose ranging from 10 mg/m² to 300 mg/m². In one embodiment, the HDAC inhibitor is administered in a dose ranging from 15 mg/m² to 250 mg/m². In one embodiment, the HDAC inhibitor is administered in a dose ranging from 20 mg/m² to 200 mg/m². In one embodiment, the HDAC inhibitor is administered in a dose ranging from 25 mg/m² to 150 mg/m². In one embodiment, the HDAC inhibitor is administered in a dose ranging from 25 mg/m² to 100 mg/m². In one embodiment, the HDAC inhibitor is administered in a dose ranging from 25 mg/m² to 75 mg/m².

In one embodiment, the HDAC inhibitor is administered orally on a daily basis. In one embodiment, the HDAC inhibitor is administered orally every other day. In one embodiment, the HDAC inhibitor is administered orally every third, fourth, fifth, or sixth day. In one embodiment, the HDAC inhibitor is administered orally every week. In one embodiment, the HDAC inhibitor is administered orally every other week. Merck’s ZOLINZA® (vorinostat) is administered 400 mg orally once daily with food.

In one embodiment, the HDAC inhibitor and the demethylating agent (and optionally a CCR2 and/or CXCR2 inhibitor) are administered intravenously. In one embodiment, the combination is administered intravenously over a 1-6 hour period. In one embodiment, the combination is administered intravenously over a 3-4 hour period. In one embodiment, the combination is administered intravenously over a 5-6 hour period. In one embodiment, the combination is administered intravenously over a 4 hour period.

In one embodiment, a combination with increasing doses of the HDAC inhibitor is administered over the course of a cycle. In one embodiment, the dose of about 8 mg/m² followed by a dose of about 10 mg/m², followed by a dose of about 12 mg/m² of the HDAC inhibitor is administered over a cycle.

In one embodiment, the HDAC inhibitor is administered intravenously and the demethylating agent (and optionally a CCR2 and/or CXCR2 inhibitor) is administered subcutaneously. In one embodiment, the HDAC inhibitor is administered intravenously and the demethylating agent (and optionally a CCR2 and/or CXCR2 inhibitor) is administered orally. In one embodiment, the HDAC inhibitor and the demethylating agent (and optionally a CCR2 and/or CXCR2 inhibitor) are administered orally.

In one embodiment, the demethylating agent is administered daily based on 7 to 14 days administration every 28-day cycle in a single or divided doses in a four to forty week period with a rest period of about a week or two weeks.

In one embodiment, the demethylating agent is administered daily and continuously for four to forty weeks at a dose of from about 10 to about 150 mg/m² followed by a break of one or two weeks. In a particular embodiment, the demethylating agent is administered in an amount of from about 0.1 to about 4.0 mg/day for four to forty weeks, with one week or two weeks of rest in a four or six week cycle.

In one embodiment, the demethylating agent is administered intravenously to patients in an amount of from about 0.1 to about 4.0 mg per day for about 7 to about 14 days followed by about 14 to about 21 days of rest in a 28 day cycle combined with the HDAC inhibitor administered intravenously in a dose of about 0.5 mg/m² to about 28 mg/m² administered on days 1, 8 and 15 of the 28 day cycle.

In one embodiment, the demethylating agent is administered intravenously to patients in an amount of from about 0.10 to about 4.0 mg per day for about 7 to about 14 days followed by about 14 to about 21 day of rest in a 28 day cycle combined with the HDAC inhibitor administered orally in a dose of about 10 mg/m² to about 300 mg/m² administered on days 1, 8 and 15 of the 28 day cycle.

In one embodiment, the demethylating agent is administered subcutaneously to patients in an amount of from about 0.10 to about 4.0 mg per day for about 7 to about 14 days followed by about 14 to about 21 day of rest in a 28 day cycle combined with the HDAC inhibitor administered intravenously in a dose of about 10 mg/m² to about 300 mg/m² administered on days 1, 8 and 15 of the 28 day cycle.

In one embodiment, the demethylating agent is administered subcutaneously to patients in an amount of from about 0.10 to about 4.0 mg per day for about 7 to about 14 days followed by about 14 to about 21 day of rest in a 28 day cycle combined with the HDAC inhibitor administered orally in a dose of about 10 mg/m² to about 300 mg/m² administered on days 1, 8 and 15 of the 28 day cycle.

In one embodiment, the demethylating agent is administered orally to patients in an amount of from about 0.10 to about 4.0 mg per day for about 7 to about 14 days followed by about 14 to about 21 day of rest in a 28 day cycle combined with the HDAC inhibitor administered orally in a dose of about 10 mg/m² to about 300 mg/m² administered on days 1, 8 and 15 of the 28 day cycle.

In one embodiment, the demethylating agent and the HDAC inhibitor (and optionally a CCR2 and/or CXCR2 inhibitor) are administered intravenously, with administration of the HDAC inhibitor occurring 30 to 60 minutes prior to the demethylating agent during a cycle of four to forty weeks. In another embodiment, the demethylating agent is administered subcutaneously and the HDAC inhibitor (and optionally a CCR2 and/or CXCR2 inhibitor) is administered by intravenous infusion. In another embodiment, the demethylating agent is administered subcutaneously and the HDAC inhibitor (and optionally a CCR2 and/or CXCR2 inhibitor) is administered orally. In yet another embodiment, the demethylating agent and the HDAC inhibitor (and optionally a CCR2 and/or CXCR2 inhibitor) are administered orally.

In one embodiment, the demethylating agent and the HDAC inhibitor (and optionally a CCR2 and/or CXCR2 inhibitor) are administered intravenously, with administration of the demethylating agent occurring 30 to 60 minutes prior to the HDAC inhibitor, during a cycle of four to forty weeks. In another embodiment, the demethylating agent is administered subcutaneously and the HDAC inhibitor (and optionally a CCR2 and/or CXCR2 inhibitor) is administered by intravenous infusion. In another embodiment, the demethylating agent is administered subcutaneously and the HDAC inhibitor (and optionally a CCR2 and/or CXCR2 inhibitor) is administered orally. In yet another embodiment, the demethylating agent and the HDAC inhibitor (and optionally a CCR2 and/or CXCR2 inhibitor) are administered orally.

In one embodiment, the demethylating agent and the HDAC inhibitor (and optionally a CCR2 and/or CXCR2 inhibitor) are administered intravenously, simultaneously, during a cycle of four to forty weeks. In another embodiment, the demethylating agent is administered subcutaneously and the HDAC inhibitor (and optionally a CCR2 and/or CXCR2 inhibitor) is administered by intravenous infusion. In another embodiment, the demethylating agent is administered subcutaneously and the HDAC inhibitor (and optionally a CCR2 and/or CXCR2 inhibitor) is administered orally. In yet another embodiment, the demethylating agent and the HDAC inhibitor (and optionally a CCR2 and/or CXCR2 inhibitor) are administered orally.

Any suitable daily dose of a CCR2 and/or CXCR2 inhibitor is contemplated for use with the compositions, dosage forms, and methods disclosed herein. Daily dose of the CCR2 and/or CXCR2 inhibitor depends on multiple factors, the determination of which is within the skills of one of skill in the art. For example, the daily dose of the CCR2 and/or CXCR2 inhibitor depends on the strength of the inhibitor. Weak CCR2 and/or CXCR2 inhibitors will require higher daily doses than moderate inhibitors, and moderate CCR2 and/or CXCR2 inhibitor inhibitors will require higher daily doses than strong inhibitors.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Cancer recurrence after surgery remains an unresolved clinical problem. ¹⁻³ Myeloid cells derived from bone marrow contribute to the formation of the premetastatic microenvironment, which is required for disseminating tumor cells to engraft distant sites.⁴⁻⁶ There are currently no effective interventions that prevent the formation of the premetastatic microenvironment.^{6,7} Here we show that, after surgical removal of primary lung, breast and esophageal cancers, low-dose adjuvant epigenetic therapy disrupts the premetastatic microenvironment and inhibits both the formation and growth of lung metastases through its selective effect on myeloid-derived suppressor cells (MDSCs). In mouse models of pulmonary metastases, MDSCs are key factors in the formation of the premetastatic microenvironment after resection of primary tumors. Adjuvant epigenetic therapy that uses low-dose DNA methyltransferase and histone deacetylase inhibitors, 5-azacytidine and entinostat, disrupts the premetastatic niche by inhibiting the trafficking of MDSCs through the downregulation of CCR2 and CXCR2, and by promoting MDSC differentiation into a more-interstitial macrophage-like phenotype. A decreased accumulation of MDSCs in the premetastatic lung produces longer periods of disease-free survival and increased overall survival, compared with chemotherapy. Our data demonstrate that, even after removal of the primary tumor, MDSCs contribute to the development of premetastatic niches and settlement of residual tumor cells. A combination of low-dose adjuvant epigenetic modifiers that disrupts this premetastatic microenvironment and inhibits metastases may permit an adjuvant approach to cancer therapy.

### Materials and Methods

No statistical methods were used to predetermine sample size. The experiments were not randomized and investigators were not blinded to allocation during experiments and outcome assessment.

Cell lines and cell culture. Lewis lung carcinoma cells (LLC1) and 4T1 cells were obtained from ATCC and cultured in RPMI 1640 medium (GIBCO) supplemented with 10% fetal bovine serum (FBS) (GIBCO). HNM007, a p53-null mouse oesophageal squamous cell carcinoma cell line transformed by HrasG12V, was provided by S. Singhal and cultured in Dulbecco’s modified Eagle’s medium (DMEM) (GIBCO) containing FBS at 10% v/v. Cells were maintained in a humidified incubator at 37° C. in the presence of 5% CO2 and passaged every 2-3 days. Cell lines were routinely tested for mycoplasma and immediately tested upon suspicion. None of the cell lines used in the reported experiments tested positive.

LLC1 and HNM007 cells were kept in RPMI 1640 medium or DMEM with a reduced (3%) serum concentration for 48 h. After that time, supernatants were collected, aliquoted and kept at -80° C. For MDSC culture, tumor supernatant (3% FBS) containing 10 ng/ml GM-CSF (tumor-conditioned medium) was used.

LLC tissue and HNM007 tissue. The LLC tissue (P3 working stock),³¹ labeled with a Pol2-Luc/GFP lentiviral vector, was provided by G. Merlino.

To generate HNM007 tissues, mice were injected subcutaneously in the right flank with 1.0×10⁵ viable HNM007 cells in 0.1 ml of PBS and Matrigel (1:1, v/v). On reaching 500-750 mm³, tumors were surgically removed, and lung metastases were monitored periodically by conebeam computed tomography (CBCT) imaging. This transplantation was referred to as passage zero (P0). The lung nodules were identified by CBCT imaging, resected and then subcutaneously transplanted into other mice (passage one (P1)). The same procedure was repeated eight times (from P2 to P9), and the lung nodules were then collected and subcutaneously transplanted into 50 mice as passage ten (P10). When the P10 tumors reached 500-750 mm³, they were collected into 100 tubes and frozen in liquid nitrogen as working stocks. All the HNM007 tumors in our studies were expanded from the P10 working stock.

Mice. Female C57BL/6 mice and BALB/c mice were purchased from Charles River Laboratories. Female mice congenic in mouse CD45 at the Ly5 locus (B.6SJL-Ptprc^{α} Pepc^{b}/BoyJ Ly5.1) were obtained from Jackson Laboratory. Female NOD/SCID/g-chain knockout (NSG) mice, bred and housed at the Johns Hopkins Animal Care Facility, were used. B6.129S4-Ccr2^{tmlIfc}/J mice were provided by S. A. McGrath-Morrow. All mice were maintained in pathogen-free conditions and used for experiments at age of 6-8 weeks. The Guide for the Care and Use of Laboratory Animals published by the National Institutes of Health (NIH publication 96-01, revised 1996) was followed, and all protocols were approved by the Johns Hopkins University Animal Care and Use Committee.

Lung spontaneous metastasis model. For preclinical studies, a vial of P3 LLC tumor was expanded subcutaneously in 10 C57BL/6 mice (equals P4). The expanded tumors were resected at 500-750 mm³ and transplanted subcutaneously into the required number of mice for the actual drug study (P5). In the HNM007 model, a vial of P10 HNM007 tumor was expanded subcutaneously in 10 C57BL/6 mice (equals P11). The expanded tumors were resected at 500-750 mm3 and transplanted subcutaneously into the required number of mice for the actual drug study (P12). For the 4T1 model, 2×10⁵ 4T1 cells were injected into the BALB/c mouse mammary fat pad using a tuberculin syringe. For all models of spontaneous lung metastasis, the primary tumors were surgically removed at 500-750 mm³, and the mice were randomized into treatment groups. For the sham surgery study, tumor-naive mice underwent a 2.0-cm skin incision and closure in right flank. Mice were treated as follows: azacitidine 0.5 mg/kg (PBS vehicle) subcutaneously injected and daily for 14 days. Entinostat 5.0 mg/kg (LLC and HNM007 models) and 2.5 mg/kg (4T1 model) (1% dimethyl sulfoxide (DMSO) in PBS vehicle) intraperitoneally injected daily for 14 days. In vivo, the CCR2 inhibitor, RS504393 (Sigma) 2 mg/kg (PBS vehicle) or RS102895 (Sigma) 2 mg/kg (PBS vehicle) was injected intraperitoneally daily for 14 days. In vivo, the NF-κB inhibitor BMS-345541 (Selleck) 30 mg/kg/d or 75 mg/kg/d (3% Tween 80 and sterile water) was orally administered daily for 3 days. The adjuvant chemotherapy regimen was paclitaxel 20 mg/kg/week injected intraperitoneally plus cisplatin 3 mg/kg, injected intraperitoneally twice a week for 2 weeks. The size of the subcutaneous tumors was measured manually and calculated by V (mm³)=0.5×L×W², in which L is length and W is width, in millimeters. The metastasis or recurrence was monitored by CBCT imaging. The time from primary-tumor resection to metastasis detection by CBCT imaging and to death was defined as the disease-free survival and overall survival period, respectively.

Xenograft studies in NSG mice. LLC tissue (P5) and HNM007 tissue (P12) were transplanted subcutaneously into the flanks of mice. Drug treatments were started 6-8 days after transplantation, when palpable tumors could be discerned. Treatment was continued for the entire duration of the study and mice were killed before tumor volumes exceeded 2,000 mm³.

CD4^{±} and CD8^{±} T cell depletion experiment. CD4 antibody (BioXcell, clone GK1.5) and CD8 antibody (BioXcell, clone 116-13.1)-mediated CD4⁺ and CD8⁺ T cell depletion in LLC mice was initiated within 24 h of primary-tumor resection. Mice were injected intraperitoneally with 500 µg of CD4 and CD8 antibodies at day 1 and day 4, respectively, after resection. As controls, mouse IgG2a isotype control (BioXcell, clone C1.18.4) and rat IgG2b isotype control (BioXcell, clone LTF-2) were injected into the control mice. CD4⁺ and CD8⁺ T cell depletion was verified by FACS analysis of peripheral blood cells.

MDSC depletion experiment. Synthetic, complementary double-stranded oligonucleotides encoding H6 peptide (TIK), and an irrelevant control peptibody (pep-irrel) (D1) were provided by L. W. Kwak.¹⁷ The recombinant peptibodies used in all in vivo studies were produced by Kempbio following an established protocol.¹⁷ After LLC tumor resection, groups of C57BL/6 mice were injected via their tail veins with 50 µg of peptibody (TIK) once per day from day 1 to day 14. Control mice received pep-irrel or PBS. MDSC depletion was verified by assessment of the MDSC population in the premetastatic lung.

CBCT guided systems. To monitor the lung metastases, the mice were subjected to CBCT imaging (laboratory of K.K.-H.W.) at different time points after primary tumor resection. The standard procedure for CBCT imaging has previously been described.³²

Drug reagents. Azacitidine (Sigma) was dissolved in distilled water at 500 µM (in vitro) and 5 mg/ml (in vivo). Entinostat (MS-275) provided by P. Ordentlich was dissolved in DMSO to concentrations of 500 µM (in vitro) and 1 mg/ml (in vivo). Both azacitidine and entinostat were aliquoted and stored at -80° C. and diluted to needed working concentrations before use. BMS-345541 (Selleck) was formulated as a 2 mg/ml solution in 3% Tween 80, water and stored at 4° C. RS504393 (Sigma) and RS102895 (Sigma) were first dissolved in DMSO and stored at -80° C. The stock solution was dissolved in normal saline at 2 mg/ml for intraperitoneal injection. All the stored reagents were for single use only.

Cell viability assays (CCK8 assay). Equal numbers of viable cells were plated in 96-well plates at the following cell seeding densities per well: LLC1 cells (2×10³), HNM007 cells (2×10³), 4T1 cells (2×10³), monocytic MDSCs (1×10⁴) and polymorphonuclear MDSCs (1×10⁴). Cells were incubated with 100 µl drug-supplemented medium, treated with DMSO (vehicle) at 0.1% or the following drug concentrations standardized to 0.1% DMSO final concentration. For LLC1 cells, HNM007 cells and 4T1 cells, the treatment regimens were: azacitidine, 25 nM, 50 nM, 100 nM, 200 nM and 400 nM; entinostat, 25 nM, 50 nM, 100 nM, 200 nM and 400 nM; combined treatment, entinostat 50 nM plus azacitidine 25 nM, 50 nM, 100 nM, 200 nM and 400 nM. After incubation for 24 h, 48 h or 72 h, cell viability was measured using a Cell Counting Kit-8 (CCK8) assay (Dojindo) according to the manufacturer’s instructions. The optical density at 450 nm (OD450 nm) was measured using a multiwell plate reader (Micro-plate Reader; Bio-Rad).

Apoptosis assay. Apoptotic assays in cultured cells or transferred cells were performed using the Annexin V-FITC apoptosis detection kit (BD Pharmingen). Assays were performed according to the manufacturer’s protocol.

T cell suppression assay. MDSCs were isolated using the mouse MDSC isolation kit, according to the manufacturer’s protocol (Miltenyi Biotec), or by flow cytometry. CD8⁺ T cells were isolated from the spleens of tumor-bearing mice by magnetic separation as previously described,³³ and then labelled with 5 µM carboxyfluorescein succinimidyl ester (CFSE) (Invitrogen). The CFSE-labelled T cells were incubated with CD11b⁺Ly6C_{low}Ly6G⁺ or CD11b⁺Ly6C^{high}Ly6G⁻ cells isolated from the lung premetastatic niche (day 3) at different ratios in a 96-well plate cultured with CD3/CD28 beads (GIBCO) at 37° C. with 5% CO₂. After 72 h, the cells and supernatant were collected. Cells were stained with anti-CD8 antibody so that CD8⁺ T cells could be specifically gated and examined, and the CFSE fluorescence intensity of the CD8⁺ T cells was determined by flow cytometry. IFNγ concentrations in the supernatant were determined by enzyme-linking immunosorbent assay (ELISA) (R&D systems) according to the manufacturer’s instructions.

In vitro transwell migration assay. Monocytic or polymorphonuclear MDSCs isolated from the bone marrow by FACS were incubated in tumor-conditioned medium in the upper chamber of transwell inserts (5-µm pore for monocytic MDSCs or 3-µm pore for polymorphonuclear MDSCs) with CCL2 (5 ng/ml or 10 ng/ml) or CXCL1 (20 ng/ml or 50 ng/ml) in the lower chamber. Transmigrated monocytic or polymorphonuclear MDSCs were enumerated following a 60-min or 120-min incubation. Fold changes were normalized to the migration of the cells in the unstimulated mock group (set at 1).

FACS profiling and sorting of immune cells. FACS profiling and sorting of immune cells were performed after both lungs were collected from LLC mice, HNM007 mice and mice of the 4T1 model (hereafter 4T1 mice) in mock-treated or low-dose-AET-treated groups. Tissue was then digested for 30 min at 37° C. in digestion buffer (RPMI, FBS (5%), collagenase type 1 (Sigma-Aldrich, 0.2%), collagenase type 2 (Sigma-Aldrich, 0.2%) and DNase I (Roche, 50 U/ml)), minced and strained through a 40-µm cell strainer to obtain a single-cell suspension. ¹⁴ Perfused livers were cut into small fragments and incubated (37° C., 250 rpm for 30 min) with 5 ml digestion buffer (5% FBS, 0.5 mg/ ml collagenase VIII (Sigma-Aldrich) and 0.1 mg/ml DNase I in PBS). This was followed by 3 cycles of washing with PBS at 400 rpm from which the supernatant was taken, omitting the parenchymal cell pellet. Spleens were mechanically dissociated, and red blood cells were lysed in 1 x lysing buffer (BD Bioscience). Femurs and tibias from mice were dissected, and the bone marrow was flushed with RPMI 1640 medium. The cells were filtered through a 70-µm cell strainer. Blood was obtained by tail vein puncture or by heart puncture after exposing the organ. Blood was collected into tubes containing 1.0 ml of PBS with 2 mM EDTA. Red blood cells were then lysed with lysing buffer and the cell pellet was washed twice in PBS. Cells were counted and then blocked with rat monoclonal anti-CD16/CD32 (Fc block antibody) in PBS for 30 min at 4° C. Cells were then stained with antibodies. For intracellular antigens, cells were fixed and permeabilized in fixation/permeabilization buffer (eBioscience) for 30 min at 4° C., washed and stained with intracellular antibodies for 30 min at 4° C. Information about all the antibodies used is provided in the published paper, https://doi.org/10.1038/s41586-020-2054-x.

Cell transfer experiments. For adoptive transfer experiments, MDSCs were sorted from the bone marrow of CD45.1⁺ LLC mice by FACS. A total of 5x10⁶ monocytic or polymorphonuclear MDSCs were transferred into a CD45.2 mouse via tail-vein injection within 24 h of resection.

Immunofluorescence analysis. Immediately after perfusion, mice lungs were embedded in optimal cutting temperature compound, snap-frozen and stored at -80° C. until analyzed. Five-micrometer cryosections were cut, air-dried, acetone-fixed and blocked with 10% normal goat serum (30 min, Sigma-Aldrich). To detect MDSCs, slides were stained with rat anti-GR1 antibody (1:500, Biolegend). To detect T cells, slides were stained with rabbit anti-CD4 antibody (1:200, Abcam) and rat anti-CD8a antibody (1:100, ebioscience). To detect GFP signal, slides were stained with rabbit anti-GFP (1:200, Abcam). Sections were washed in PBS 3 times before adding goat anti-rabbit Alexa Fluor 488 and goat anti-rat TRITC secondary antibodies in blocking solution for 45 min at room temperature. All sections were mounted in ProLong Gold Antifade Mountant with DAPI (Thermo Fisher Scientific) and imaged with a Nikon Confocal Microscope C1 and analyzed with EZ-C1 software (Nikon).

Gene-expression analysis. For genome-wide gene expression analysis, we used gene-expression arrays at the Sidney Kimmel Comprehensive Cancer Center Microarray Core at the Johns Hopkins University. MDSCs were isolated from bone marrow or lungs by FACS. RNA preparations were obtained from monocytic MDSCs from lung or bone marrow pooled from 6-8 mice per sample (low-dose AET group) or from 1-2 mice per sample (vehicle-treatment group). After total cellular RNA was extracted using the TRIzol method (Life Technologies), RNA concentration was determined using the NanoDrop machine and software (Thermo Fisher Scientific). Around 400 ng of total RNA was used to generate cDNA with the Quanti-Tect Reverse Transcription Kit (Quanta Biosciences). Transcriptomic profiles were obtained using Agilent 4x44K mouse Gene Expression v.2 arrays following the manufacturer’s instructions. Microarray data were analyzed with the R package limma as described in the manual. In brief, background signals were corrected using the normexp method (with offset=50). Then, normalization within arrays and between arrays was performed using the losses and Aquantile methods. The differential gene expression was defined as log2-transformed fold change > absolute (0.5) and an FDR adjusted P < 0.05. Ranked lists of log2-transformed fold change were analyzed using GSEA by the Broad-Institute-developed data packages.³⁴ Significantly enriched gene sets were defined using an FDR cut-off of <0.25. P values were defined as <0.05 when comparing treated conditions versus controls.

DAVID analysis of median absolute deviation derived genes. Gene Ontology and Kyoto Encyclopedia of Genes and Genomes (KEGG) pathway enrichment analyzes were conducted using the DAVID Bioinformatics resources database.^{35,36} Only categories that were below the FDR adjusted P value of 0.05 were considered.

Quantitative real-time PCR. Quantitative real-time PCR was performed with SYBR Green I detection chemistry (Bio-Rad Laboratories) using the Applied Biosystems 7500 Fast Real-Time PCR System and its software. β-Actin was used as a reference gene. The specific primers used for quantitative real-time PCR are listed in the online paper). The ΔΔCt method was used to calculate relative expression levels.

Immunoblotting. Protein was extracted by RIPA buffer containing protease and phosphatase inhibitors. Six-to-ten per cent Bis-Tris protein gels were equally loaded with 30 µg protein, electrophoresed at 110 V, and electrotransferred to PVDF membranes. Membranes were blocked with 10% milk in TBST and immunoblotted with the following antibodies: rabbit polyclonal anti-MMP-9 (Abcam, 1:1,000), rabbit monoclonal anti-TGFβ (Abcam, 1:1,000), rabbit polyclonal anti-ARG1 (Abcam, 1:1,000), rabbit monoclonal anti-VEGF-A (Abcam, 1:1,000), rabbit monoclonal anti-S100A8 (Abcam, 1:1,000), rabbit monoclonal anti-TNF (Abcam, 1:1,000), rabbit monoclonal anti-IL-6 (Cell Signaling Technology, 1:1,000), rabbit polyclonal anti-DNMT1 (Cell Signaling Technology, 1:1,000), rabbit monoclonal anti-EGR1(Abcam, 1:1,000), rabbit monoclonal anti-EGR2 (Abcam, 1:1,000), rabbit monoclonal anti-PPARγ (Cell Signaling Technology, 1:1,000), mouse monoclonal anti-MAF-B (Santa Cruz Biotechnology, 1:100), rabbit monoclonal antip50 (Cell Signaling Technology, 1:1,000), rabbit monoclonal anti-p52 (Cell Signaling Technology, 1:1,000), rabbit monoclonal anti-RELB (Cell Signaling Technology, 1:1,000), rabbit monoclonal anti-p65 (Cell Signaling Technology, 1:1,000), mouse monoclonal anti-β-actin (Sigma Aldrich, 1:10,000). The loading control antibodies (anti-β-actin) in all cases were applied. Information about all the antibodies used is provided in the online paper.

NF-κB DNA-binding capability assay. To measure NF-κB activation, the TransAM NF-κB Family Transcription Factor Assay Kit (43296, Active Motif) was used according to manufacturer’s protocol. In brief, bone-marrow-derived monocytic MDSCs from LLC mice treated with vehicle or low-dose AET were isolated, and nuclear extracts were prepared in lysis buffer AM2. Nuclear lysates were incubated with oligonucleotides containing the NF-kB-binding consensus sequence, and specific antibodies were used to detect the different subunits within the bound complexes. Quantification was performed via colorimetric readout of absorbance at 450 nm.

H&E staining and imaging. For histological analysis, lungs were fixed in 10% formalin overnight, and subsequently transferred into 70% ethanol, embedded in paraffin according to standard protocols. Sections (5 µm) were stained with H&E and viewed under the Nikon Eclipse NiE microscope (Nikon Instruments). Images from the whole slide were acquired by Nikon Nis Element software. For each sample, sections from three levels were analyzed. The number and area of metastatic sites were quantitated using Aperio Imagescope software.

Adjuvant epigenetic treatment trial. The J1037 (NCT01207726) study was a randomized phase-II study that compared the low-dose AET (5-azacytidine plus entinostat) with standard of care (observation) in patients with stage I (T1-2aN0) NSCLC after primary tumor resection. The J1037 study was performed in full accordance with the guidelines for Good Clinical Practice and the Declaration of Helsinki, and all patients gave written informed consent. Protocol approval was obtained from the Johns Hopkins Hospital and Anne Arundel Medical Center Ethics Committee. An independent data monitoring committee reviewed the safety data. The patients from the adjuvant epigenetic treatment group received the combination of azacitidine at 40 mg/m² on days 1-5 and 8-10 with entinostat at a 7-mg fixed dose on days 3 and 10 of each 28-day cycle. The primary end point was the effect of 5-azacytidine plus entinostat on the hazard of 3 years of progression-free survival in patients with resected stage I non-small-cell lung cancer. Finally, 13 patients were enrolled in the trial. Owing to the difficulty in enrolling patients, the trial was prematurely terminated on 1 May 2015.

Statistical analysis. Flow and imaging data were collected using FlowJo Version 10.0.7, or Summit Version 5.4 (Beckman Coulter). All the experiments were performed in biological and technical triplicates. Values reported in figures are expressed as the standard error of the mean, unless otherwise indicated. Depending on the type of experiment, data were tested using two-sample, two-sided t-test, two-sided log-rank test, one-way analysis of variance (ANOVA), hypergeometric test or Mann-Whitney U-test. P values of < 0.05 were considered significant. *P < 0.05, **P < 0.01, ***P < 0.001. Statistical analyzes were performed with GraphPad Prism 7.0 (GraphPad Software) or R version 3.6.1.

### Results

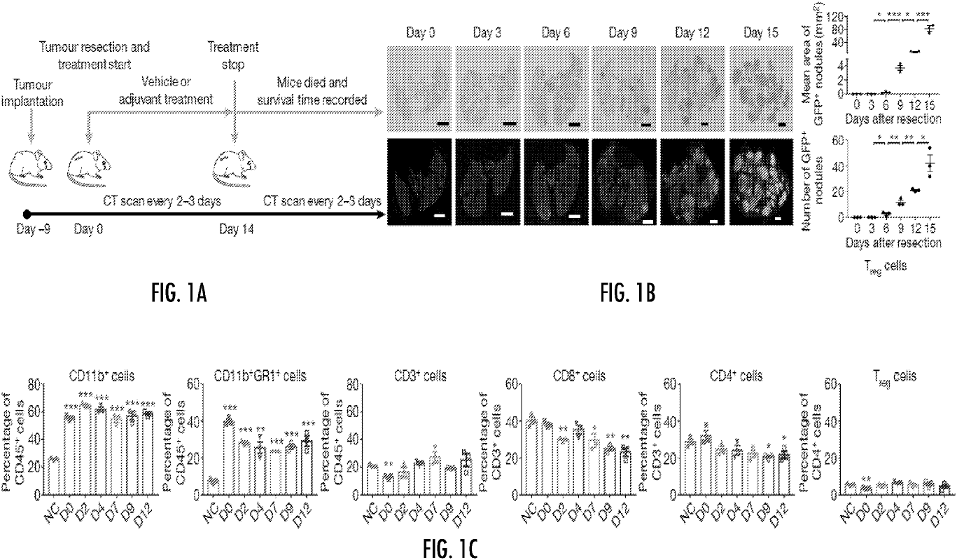

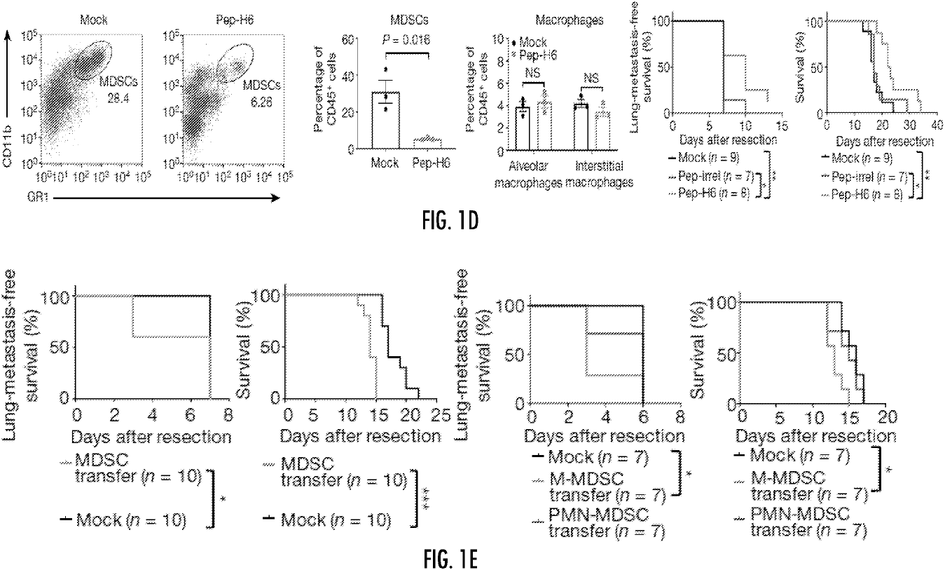

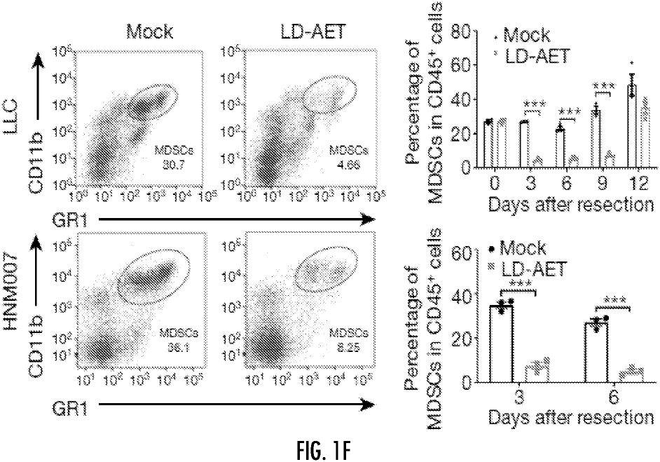

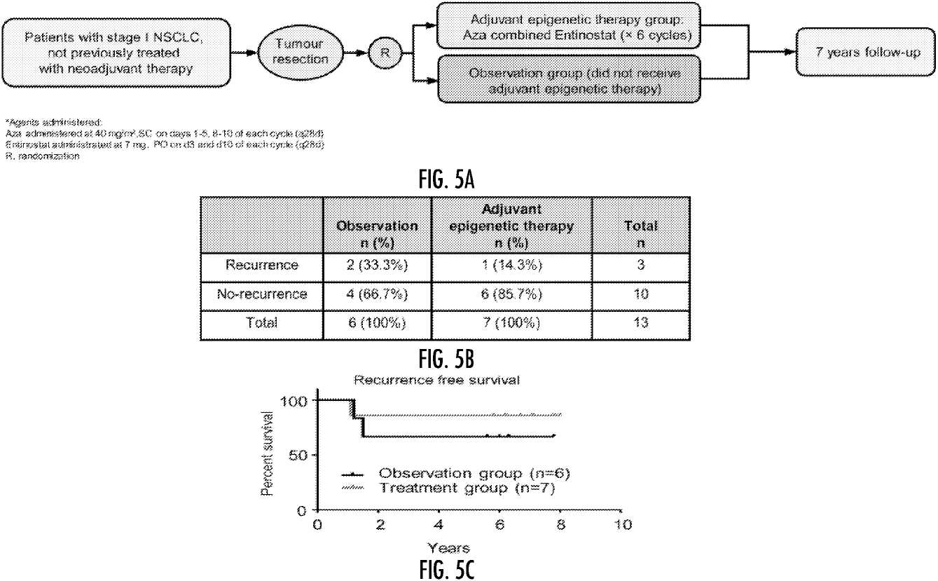

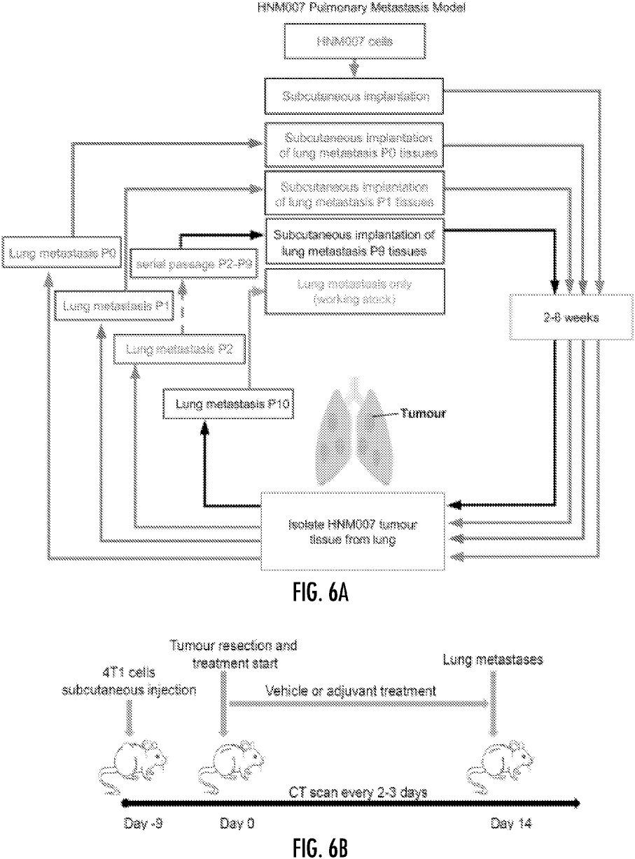

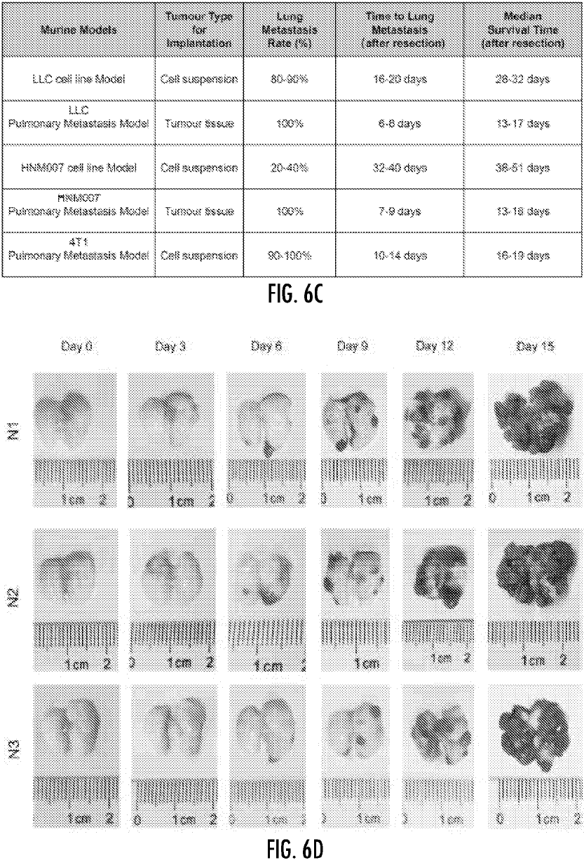

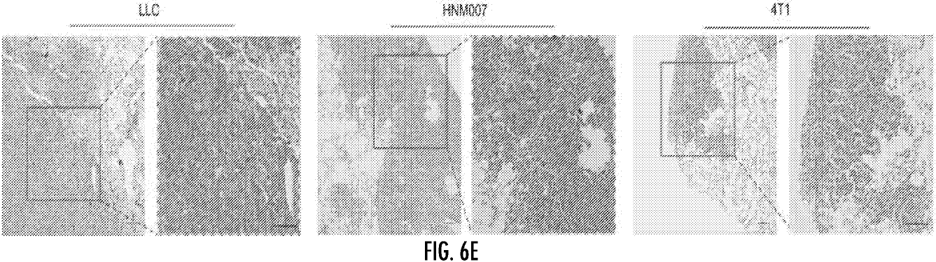

MDSCs delineate premetastatic niches. In 2011, we initiated a randomized, phase-II adjuvant clinical trial using low-dose 5-azacytidine and entinostat in patients with stage-I (T1-2aN0) NSCLC. The trial was prematurely terminated after 13 patients enrolled, owing to the requirement for 5-azacytidine to be given in an outpatient clinic. Nevertheless, patients tolerated the therapy well with a low rate of postsurgical recurrence (14.3% versus 33.3%), which suggested that epigenetic therapy may decrease relapses after curative surgery (FIGS. 5A-5C). A previous study found that combined epigenetic modifiers blunt metastases in a mouse model of aggressive NSCLC with an immune-competent microenvironment14. Here we reveal a key mechanism of action of adjuvant epigenetic therapy (AET) on premetastatic niches that could underlie these previous findings. Low-dose AET modulates innate immune factors in the lung microenvironment to inhibit tumor recurrence after resection in three syngeneic models of aggressive pulmonary metastasis: Lewis lung carcinoma (LLC), HNM007 oesophageal squamous cell carcinoma (mice of the LLC and HNM007 models have no extrapulmonary metastases) and 4T1 mammary cancer (FIG. 1A, FIGS. 6A-6E).

It is known that the microenvironments of distant organs—the targets of future metastases—are not passive receivers of circulating tumor cells, but instead are selectively modified by the primary tumor before metastasis.^{6,7,15,16} Settlement of tumor cells at distant sites is dependent on tumor-secreted factors and tumor-shed extracellular vesicles that enable the premetastatic microenvironment to support their colonization.^{6,7,15,16} To study whether low-dose AET affects the dynamics of the premetastatic microenvironment, we used mouse models of highly aggressive, pulmonary metastasis that exhibit pulmonary metastases in 90-100% of mice after resection, with median disease-free survival times of fewer than 14 days (FIGS. 1A, 1B, FIGS. 6A-6D).

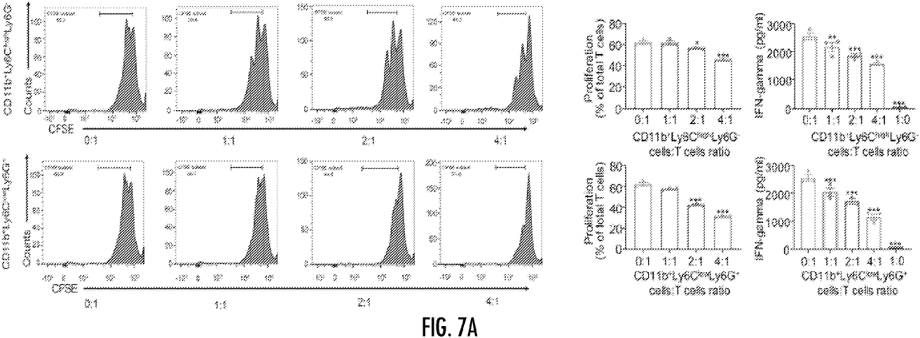

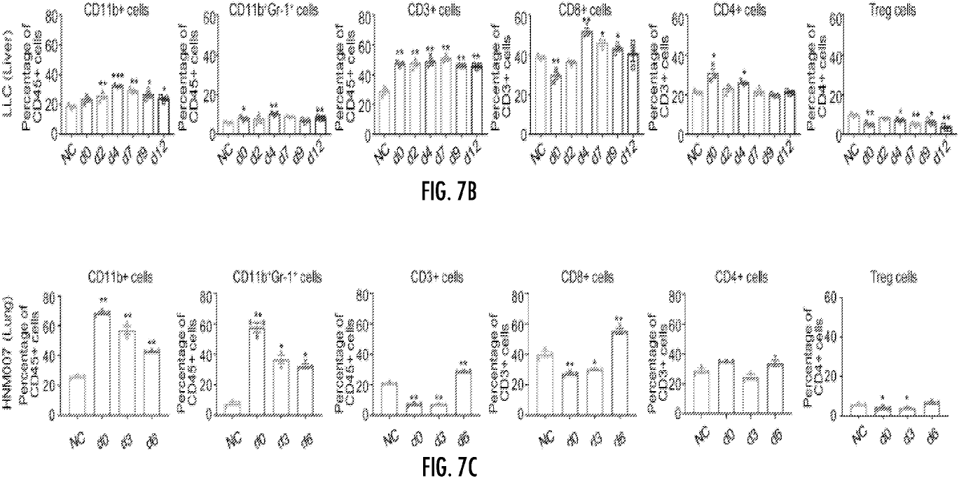

In mice of the LLC model (hereafter LLC mice), no tumor-cell infiltration of the lung was detectable by haematoxylin and eosin (H&E) and no green fluorescent protein (GFP)⁺ tumor cells were observed by immunofluorescence staining until day 6 after resection (FIG. 1B). By contrast, CD11b⁺ cells-including CD11b⁺GR1⁺ cells—are already substantially increased in the lung on the day of resection (day 0) (FIG. 1C). CD11b⁺GR1⁺ cells collected from the lungs on day 3 after primary tumor resection (before visible metastases), inhibited both T cell proliferation and activation in vitro, which suggests that recruited CD11b⁺GR1⁺ cells in the lungs are functional MDSCs (FIG. 7A). These MDSCs were the most-increased immune component of all CD11b⁺ cells in tumor-bearing mice immediately before resection. Notably, even after resection, these MDSCs in the LLC and HNM007 models persisted until day 12 in the lung-but not in the liver—as the predominant immune cells, compared to non-tumor-bearing mice (FIG. 1C, FIGS. 7B, 7C). We then used pep-H6, a peptibody that selectively targets MDSCs and has a minimal effect on other immune components, to deplete MDSCs17 (FIG. 1D). Peptibody-mediated depletion of MDSCs resulted in an increase in both disease-free survival and overall survival of the mice (FIG. 1D). By contrast, intravenous injection of MDSCs isolated from the lung of day-3 LLC mice induced metastases sooner and resulted in shorter overall survival times (FIG. 1E), indicating that these MDSCs had an important role in metastasis in this system.

Our results indicate that the accumulation of MDSCs in the lungs preceded metastasis development and delineated premetastatic niches. To test the differential role of monocytic (CD11b⁺Ly6C^{high}Ly6G⁻) and polymorphonuclear (CD11b⁺Ly6C_{low}Ly6G⁺) MDSCs in the formation of lung metastases, we transferred monocytic or polymorphonuclear MDSCs from the bone marrow of LLC mice. We found that—compared to vehicle-transferring monocytic MDSCs (but not polymorphonuclear MDSCs) resulted in a higher rate of lung metastasis at day 3 in LLC mice, which suggests that monocytic MDSCs have a predominant role in establishing the premetastatic microenvironment (FIG. 1E).

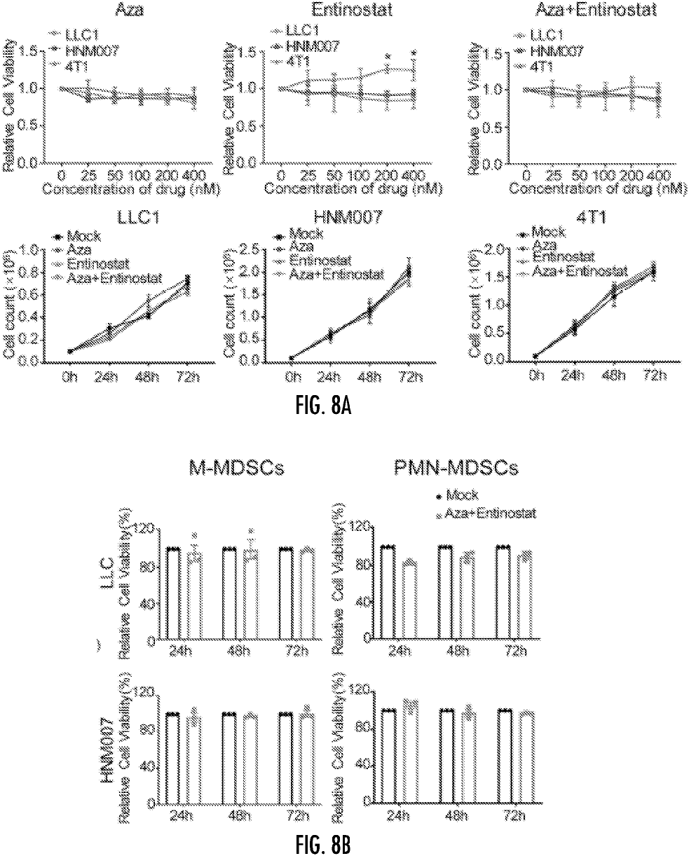

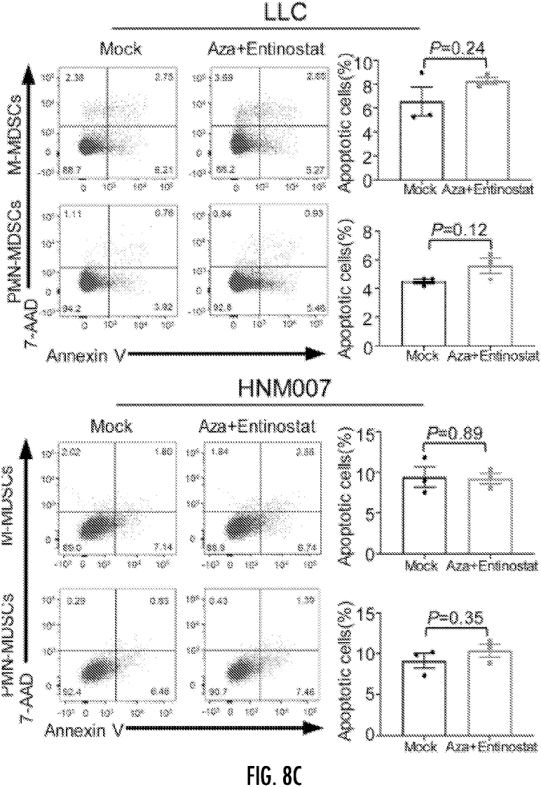

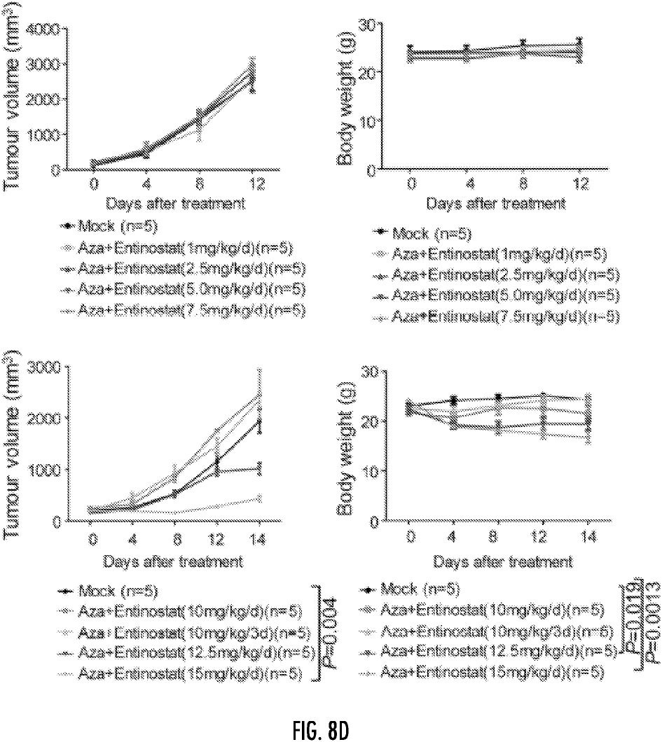

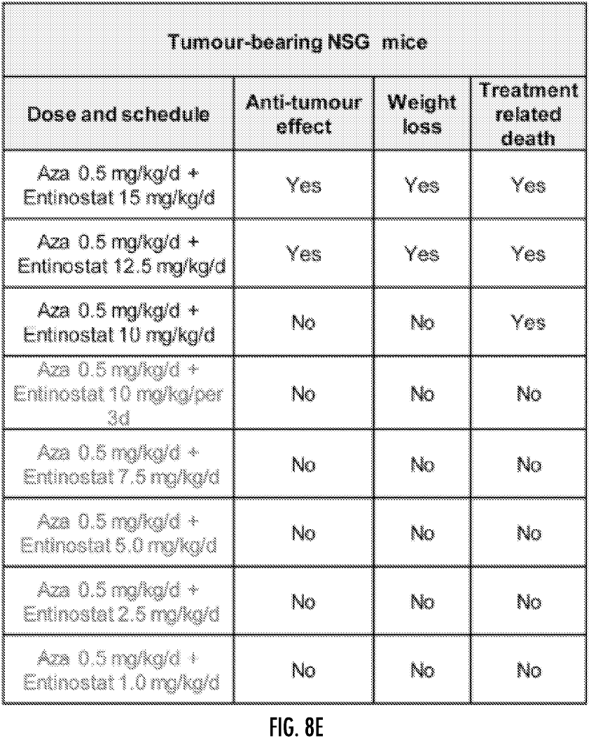

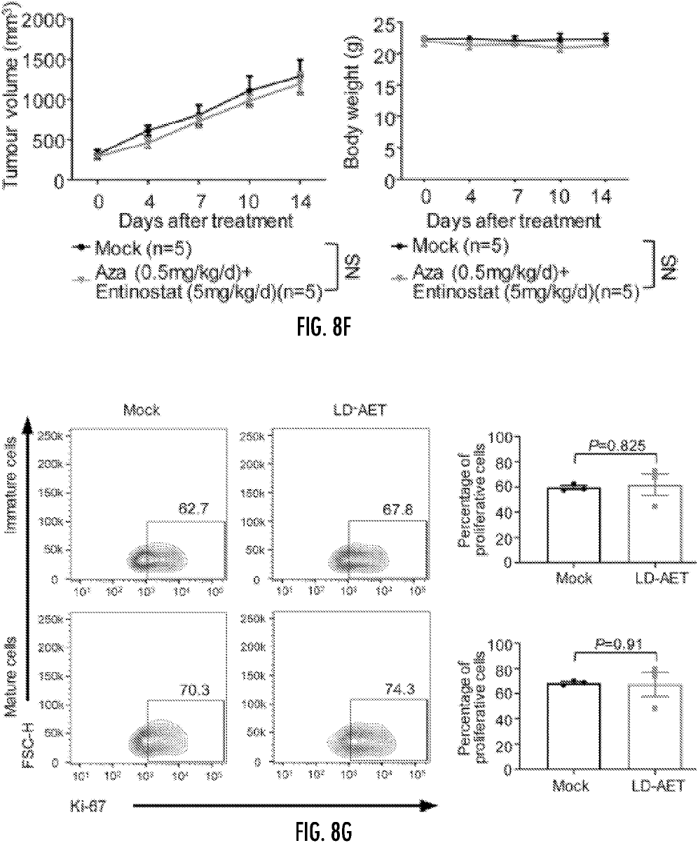

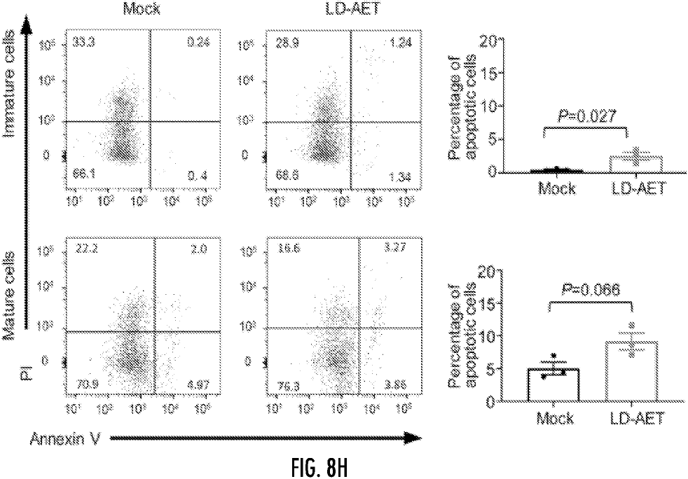

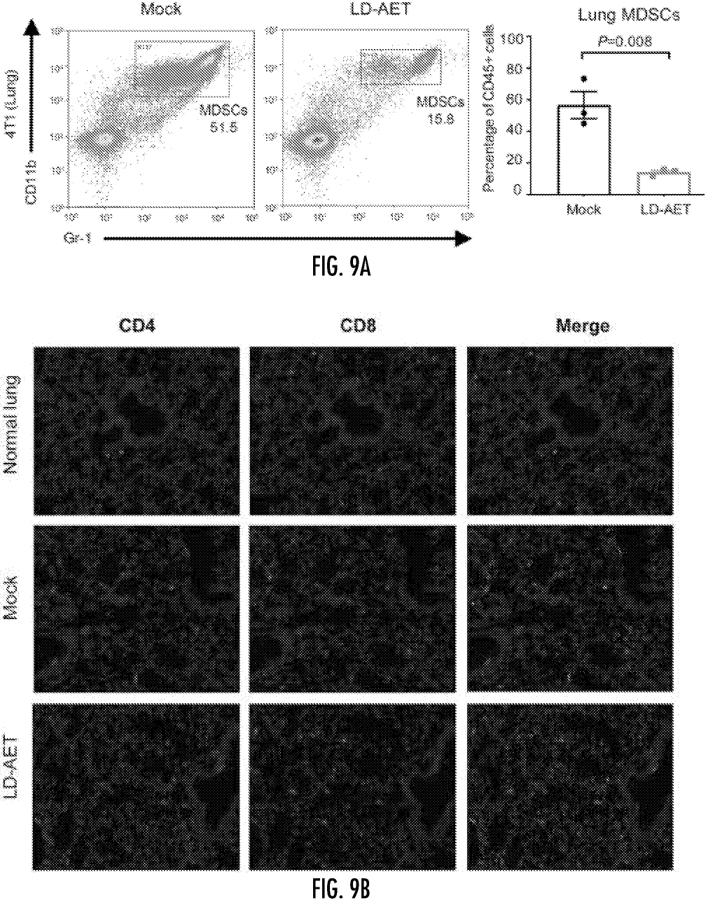

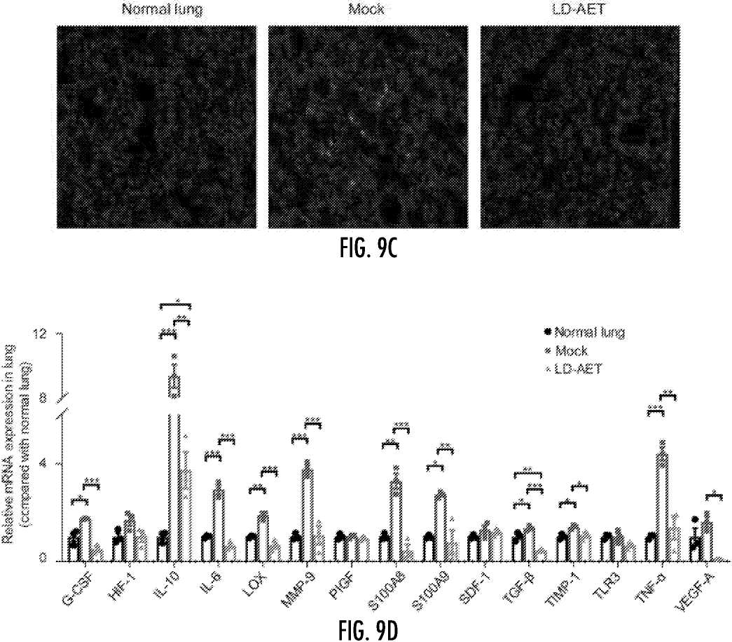

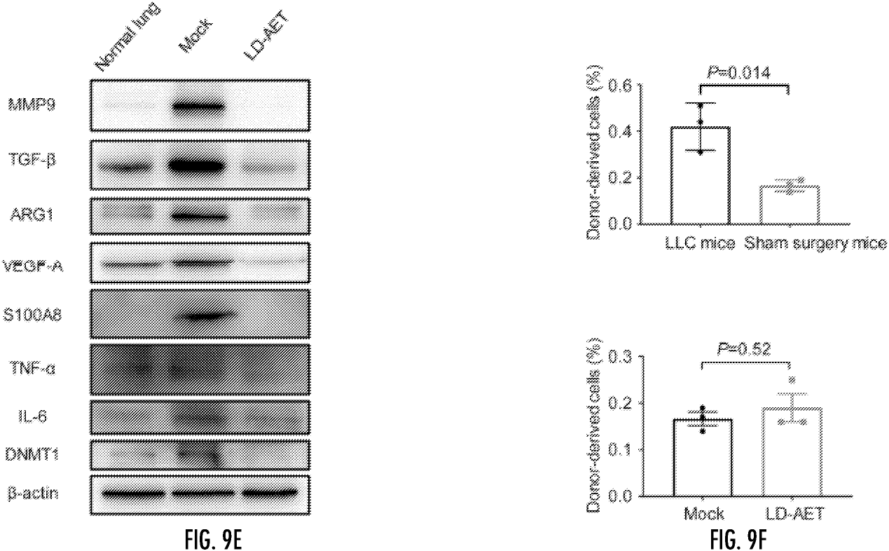

Low-dose AET impedes migration of MDSCs. It has recently been found that low doses of 5-azacytidine and entinostat are well-tolerated and clinically effective in heavily pretreated patients with NSCLC18, and that these treatments target MDSCs,¹⁹′²⁰ Low-dose 5-azacytidine (100 nM) and entinostat (50 nM) in vitro had limited effect on the proliferation of LLC1, HNM007 and 4T1 cells (FIG. 8A). Similarly, these doses did not influence the viability or apoptosis of MDSCs sorted from the bone marrow of LLC mice and mice of the HNM007 model (hereafter HNM007 mice) (FIGS. 8B, 8C). We determined the in vivo doses of 5-azacytidine and entinostat (0.5 mg per kg body weight per day and 5 mg per kg body weight per day, respectively) that had no effect on primary tumor growth and that did not cause weight loss in immune-compromised mice with LLC and HNM007 tumors (FIGS. 8D-8F). These doses also had a limited effect on the proliferation and apoptosis of CD45.1⁺ donor cells in vivo (FIGS. 8G, 8H). Importantly, in our mouse models, these doses decreased MDSCs and niche-promoting molecules in the premetastatic lung (FIG. 1F, FIGS. 9A-9E). Moreover, the percentage of donor CD45.1⁺ MDSCs in the lung is not affected by low-dose AET in the control sham-surgery mice (tumomaive recipient mice) (FIG. 9F). On the basis of these findings, we hypothesize that low-dose AET can inhibit the accumulation of MDSCs in the lung and prevent the formation of premetastatic niches in our models of pulmonary metastasis.

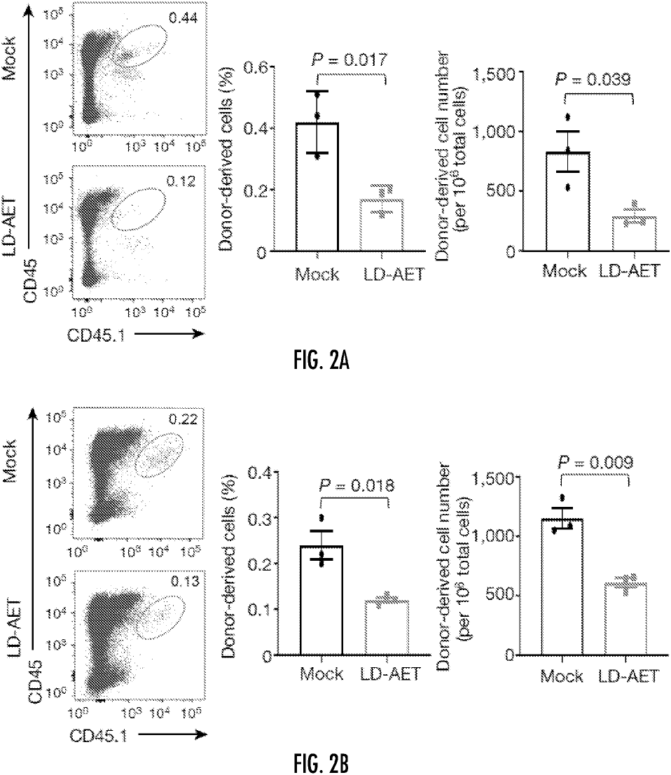

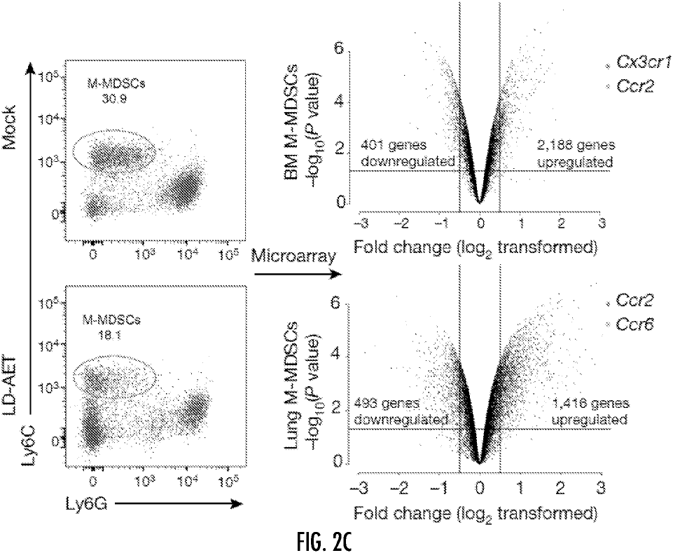

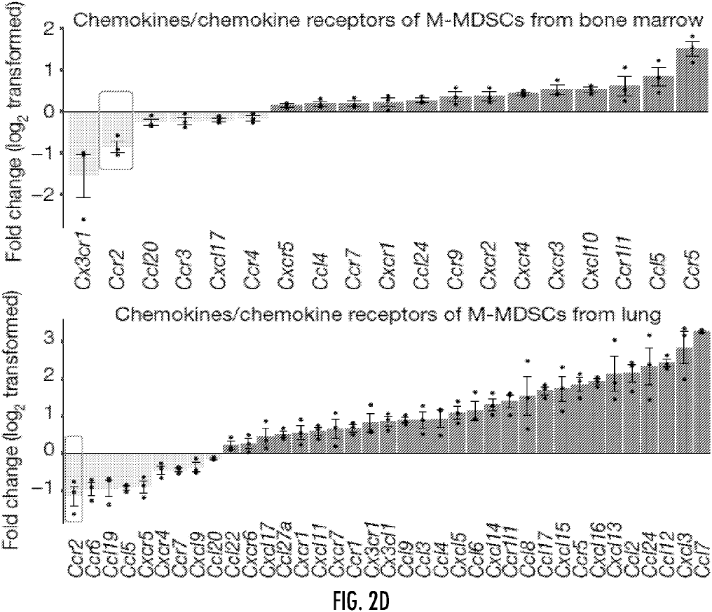

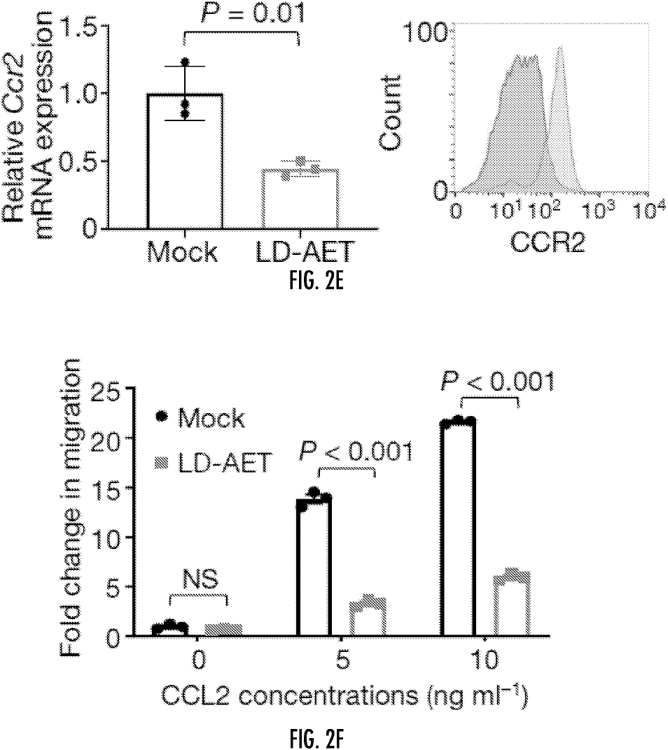

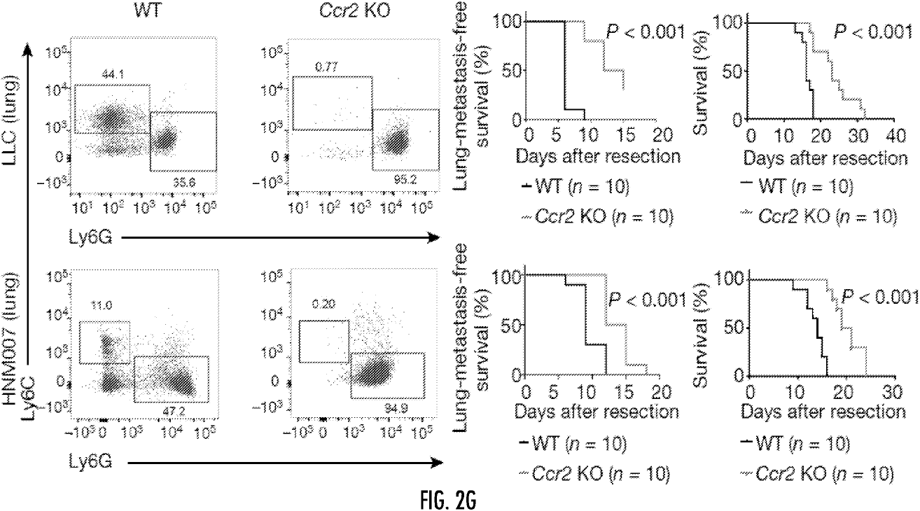

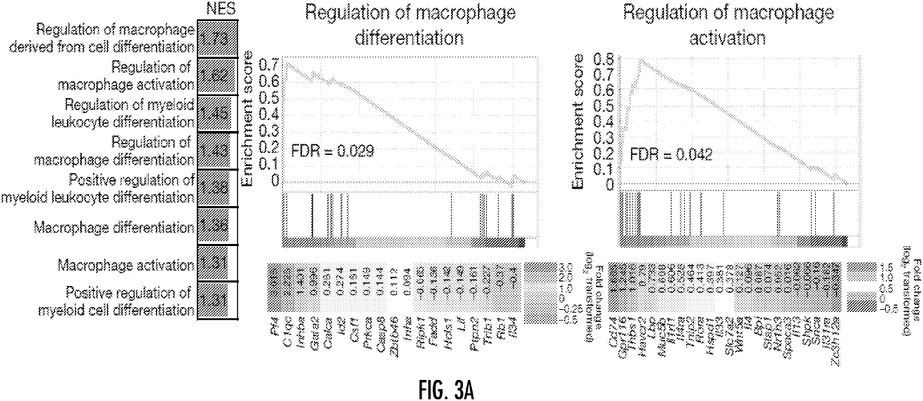

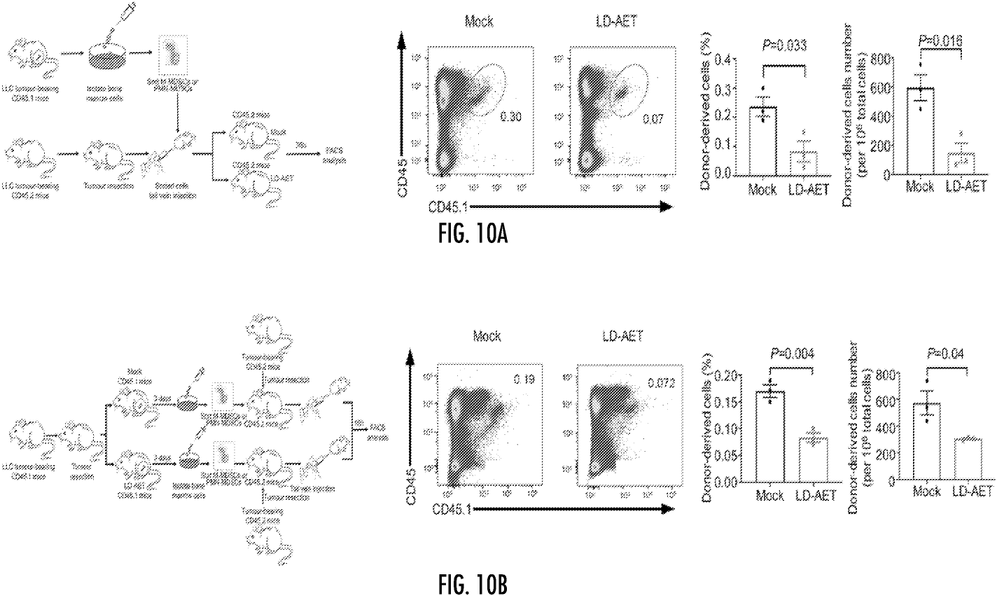

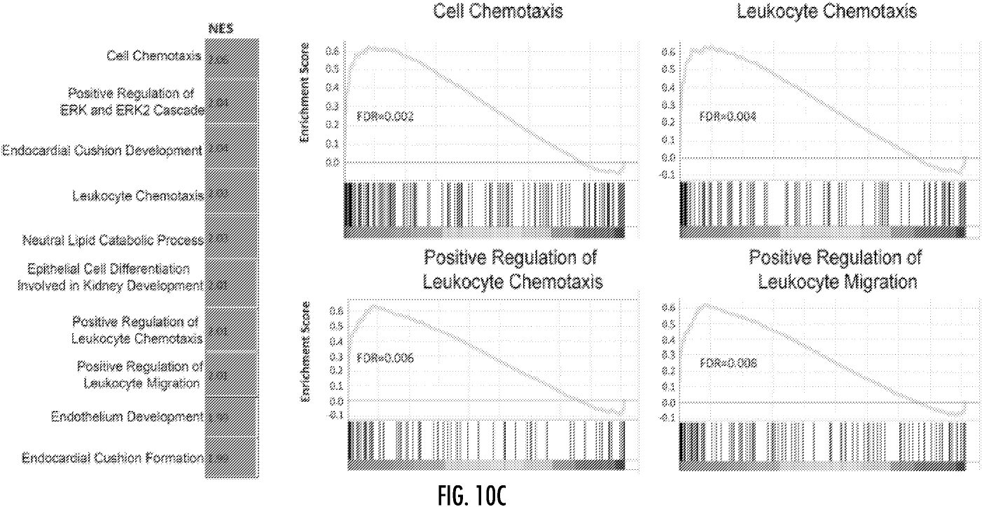

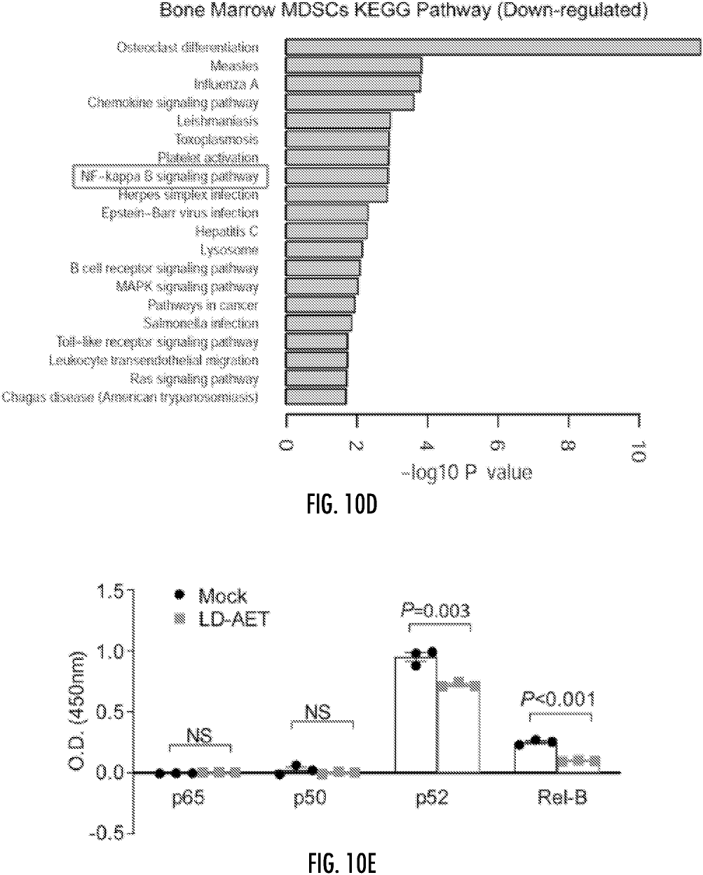

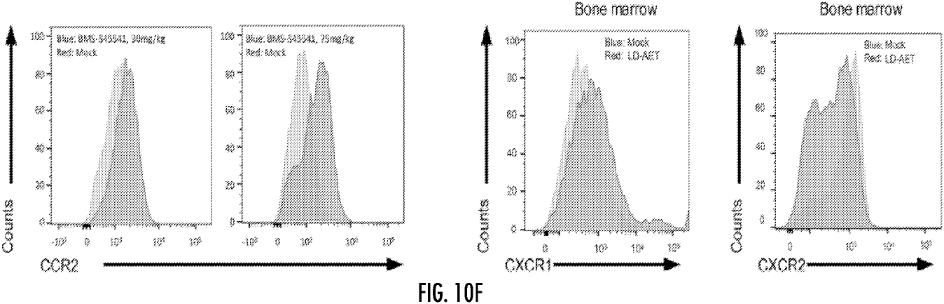

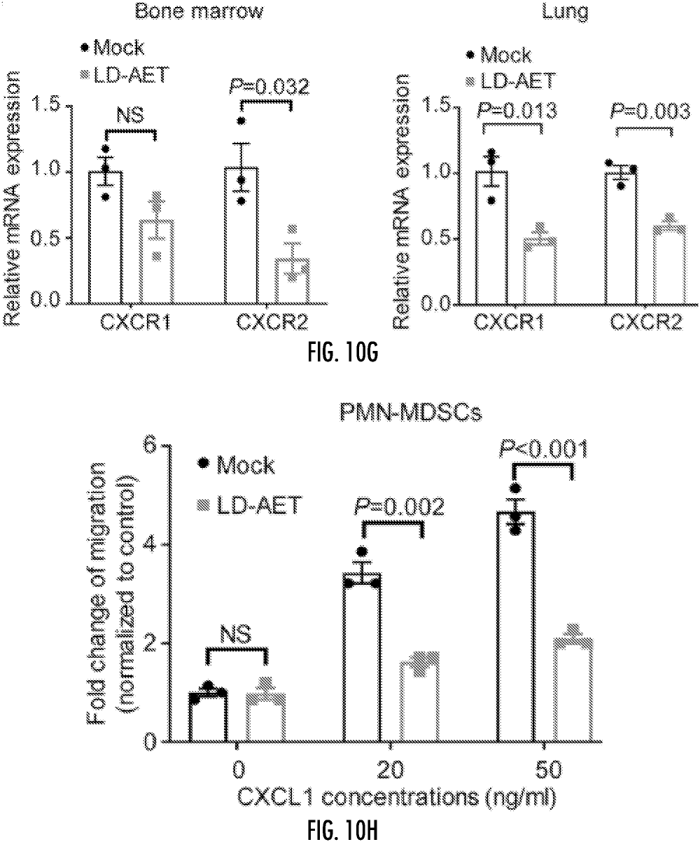

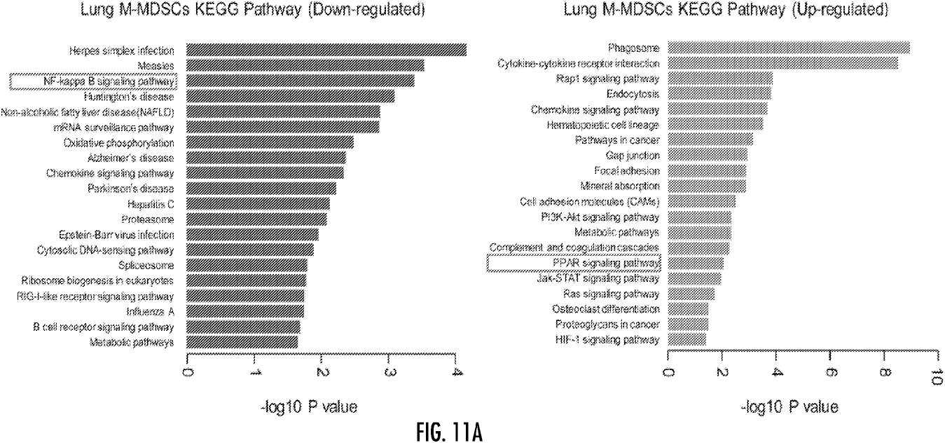

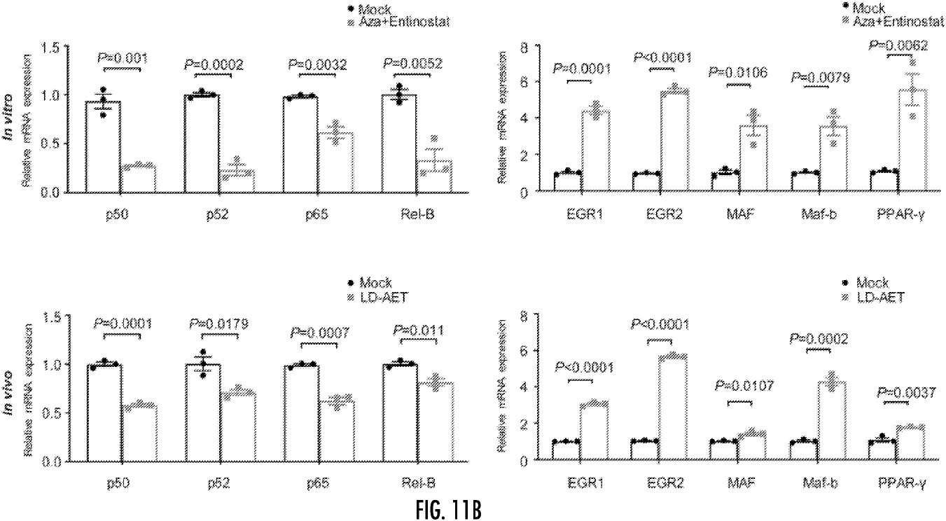

When CD45.1⁺ monocytic or polymorphonuclear MDSCs (5x10⁶ cells each) were adoptively transferred on day 0 to CD45.2 mice, 36 h after transfusion CD45.1⁺ cells decreased by 40-80% in the lungs of recipient mice treated with low-dose AET (FIG. 2A, FIG. 10A). When the same numbers of CD45.1⁺ monocytic or polymorphonuclear MDSCs isolated from bone marrow of mock-(day 3) and low-dose AET-treated mice (day 3) were adoptively transferred into CD45.2 mice on day 0, 18 h after transfusion there were significantly fewer CD45.1⁺ cells from mice treated with low-dose AET than from vehicle-treated mice in the lungs of CD45.2⁺ recipient mice, as expected (FIG. 2B, FIG. 10B). These results demonstrate that low-dose AET impedes the migration of MDSCs to the premetastatic microenvironment in the LLC model. Together with our finding that only the transfusion of monocytic (and not of polymorphonuclear) MDSCs increases lung metastases (FIG. 1E), these results showed that—although low-dose AET impairs the migration of both monocytic and polymorphonuclear MDSCs—the role of low-dose AET in targeting the trafficking of monocytic MDSCs may be more important than its targeting of polymorphonuclear MDSCs in our LLC model.

To identify differences in MDSCs from the lung and bone marrow of mock-and low-dose-AET-treated mice on day 3 after resection, we compared the gene expression of monocytic MDSCs (excluding differentiated MHC-II⁺ and F4/80⁺ macrophages) sorted from these two groups in LLC mice (FIG. 2C). Gene-set enrichment analysis (GSEA) of monocytic MDSCs from the lung showed that low-dose AET induced a substantial change in gene sets associated with immune-cell chemotaxis and migration (FIG. 10C). CCR2 expression in monocytic MDSCs from both the bone marrow and lung was significantly downregulated in the low-dose-AET group (FIG. 2D). Because CCR2 is a key regulator of monocytic cell migration from the bone marrow to the tumor microenvironment,^{5,21} these data suggest that low-dose AET may affect the trafficking of monocytic MDSCs to the premetastatic lung at least in part by downregulating CCR2.

Quantitative PCR and flow cytometry confirmed that both messenger RNA (mRNA) and protein levels of CCR2 in monocytic MDSCs from bone marrow decreased after low-dose AET (FIG. 2E). Monocytic MDSCs from bone marrow collected on day 3 from LLC mice that were treated with low-dose AET show reduced migration in a transwell assay after induction with CCL2 (FIG. 2F). Both the absolute number and percentage of monocytic MDSCs in the lung premetastatic microenvironment are negligible in Ccr 2-knockout mice, and differed from wild-type mice (FIG. 2G). Compared to the wild-type C57BL/6 mice, Ccr2-knockout mice have a longer disease-free survival and overall survival both in the LLC and HNM007 models (FIG. 2G).

We next tested the mechanism of action of low-dose AET on CCR2 expression in monocytic MDSCs from bone marrow. Database for Annotation, Visualization and Integrated Discovery (DAVID) pathway analysis reveals that the activity of the NF-_{K}B signaling pathway was significantly downregulated in bone-marrow monocytic MDSCs from LLC mice that have been treated with low-dose AET (FIG. 10D). In monocytic MDSCs from bone marrow, low-dose AET resulted in a highly significant reduction in RELB and p52 activation, compared to that found in mock-treated mice (FIG. 10E). There was a limited effect on p50 and p65 activation. Furthermore, three days of treatment with BMS-345541 (a highly selective IKB kinase (IKK) allosteric site inhibitor) resulted in decreased expression of CCR2 in monocytic MDSCs from bone marrow in vivo (FIG. 10F). Although we cannot rule out a direct effect of low-dose AET on CCR2 expression (as well as other signaling pathways), our findings suggest that low-dose AET treatment may affect—at least in part— the expression of CCR2 in monocytic MDSCs from bone marrow via the modulation of the noncanonical NF-_{K}B pathway.²²

CXCR2 and CXCR1 are known to be important for their role in trafficking polymorphonuclear MDSCs from the bone marrow to the tumor microenvironment.^{23,24} We found that CXCR2 is downregulated in polymorphonuclear MDSCs from both the bone marrow and lung by low-dose AET in the LLC model (FIG. 10G). The migration of polymorphonuclear MDSCs from the bone marrow of mice treated with low-dose AET is significantly decreased after induction with CXCL1 in a transwell migration assay (FIG. 10H). Thus, low-dose AET may inhibit the trafficking of both monocytic and polymorphonuclear MDSCs from the bone marrow to the premetastatic microenvironment by downregulating CCR2 and CXCR2 expression, respectively.

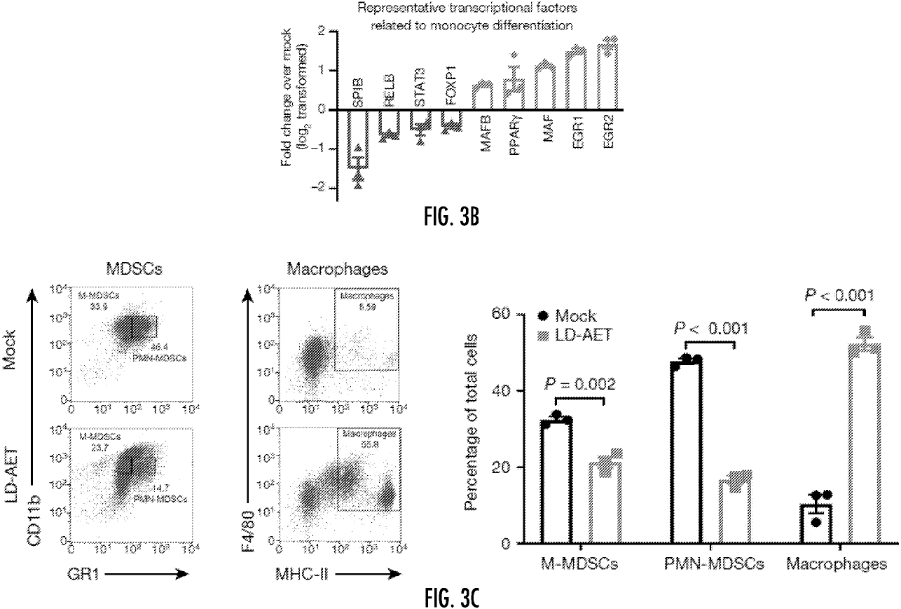

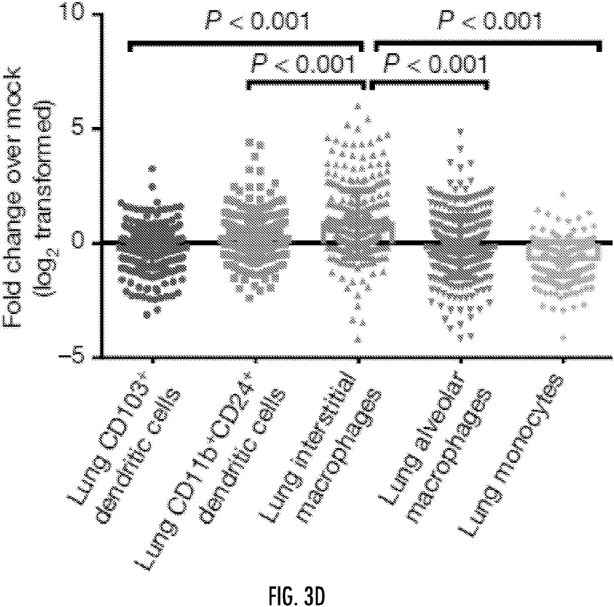

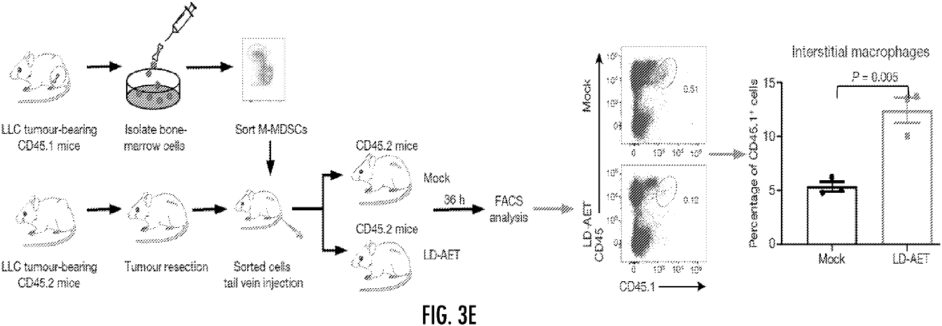

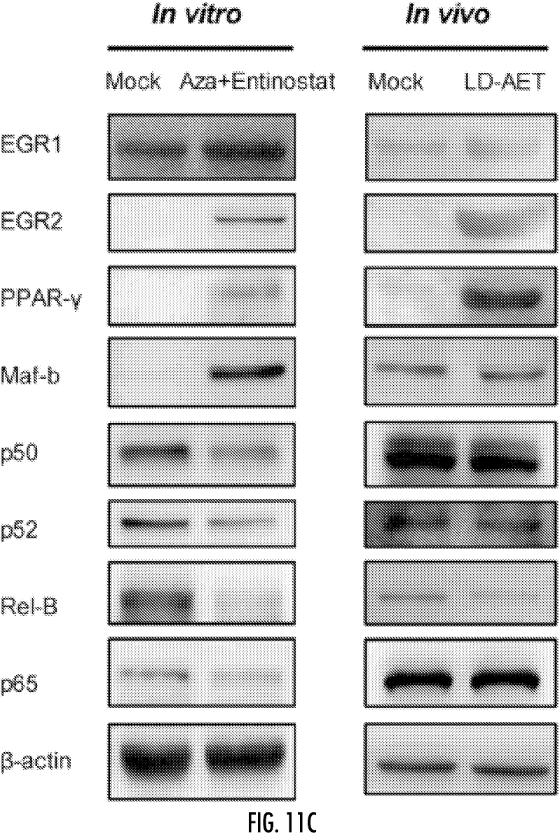

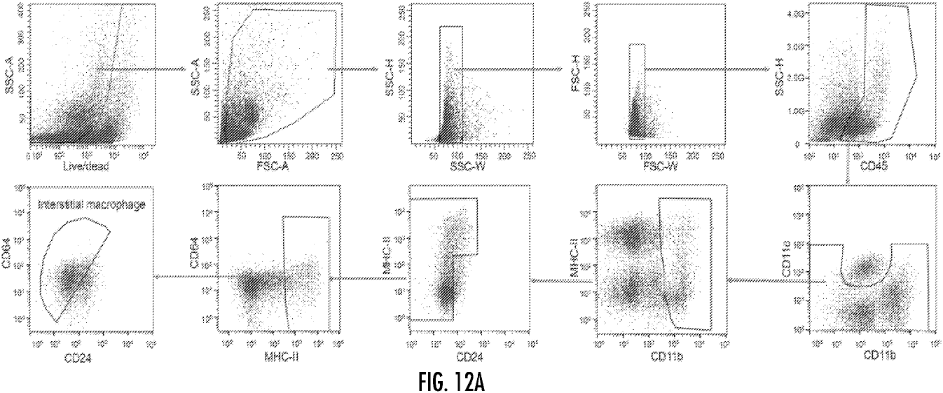

Skewing monocytic MDSC differentiation. Further query of the GSEA-derived data indicated that gene sets related to macrophage or myeloid differentiation and activation were upregulated in monocytic MDSCs from the lungs of LLC mice treated with low-dose AET (FIG. 3A). Moreover, DAVID pathway analysis reveals that the NF-κB and PPAR signaling pathways were significantly downregulated and upregulated, respectively (FIG. 11A). Consistent with differential regulation of these pathways occurring during the monocyte-to-macrophage differentiation,²⁵ there is a preferential increase in transcription factors associated with monocytic differentiation that are mainly macrophage-related (EGR1, EGR2, MAFB, MAF and PPARγ),^{25,26} and not in those that are dendritic-cell-related (SPIB, RELB, STAT3 and FOXP1)²⁶′²⁷ (FIG. 3B). The related factors have also been validated in vitro and in vivo by quantitative PCR and western blot analyzes (FIGS. 11B, 11C). In vitro epigenetic treatment of splenic monocytic MDSCs from mice bearing LLC tumors decreased the percentage and absolute number of monocytic MDSCs significantly, whereas those of macrophages increased significantly (FIG. 3C)—implying that low-dose AET might promote the differentiation of monocytic MDSCs into macrophages.

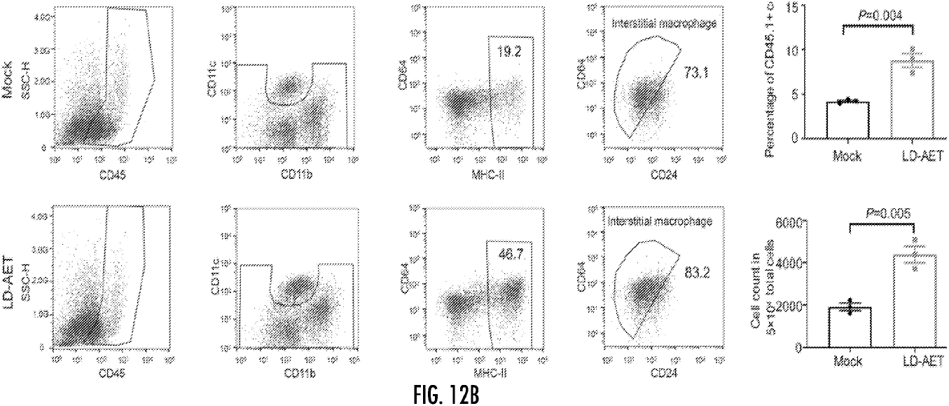

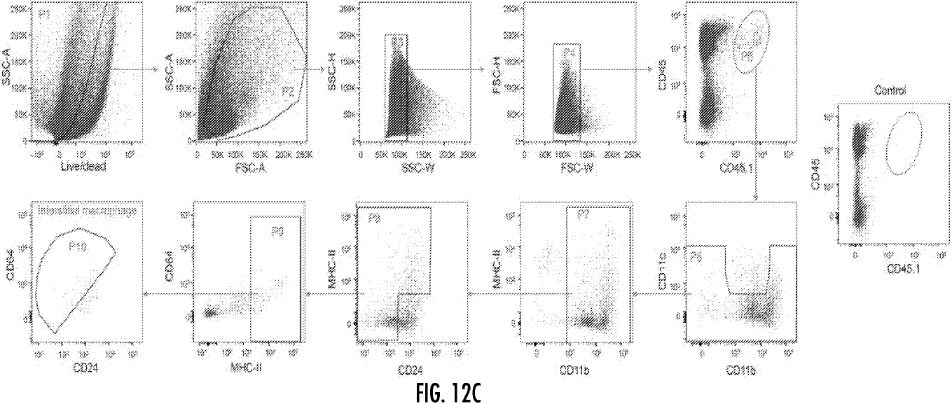

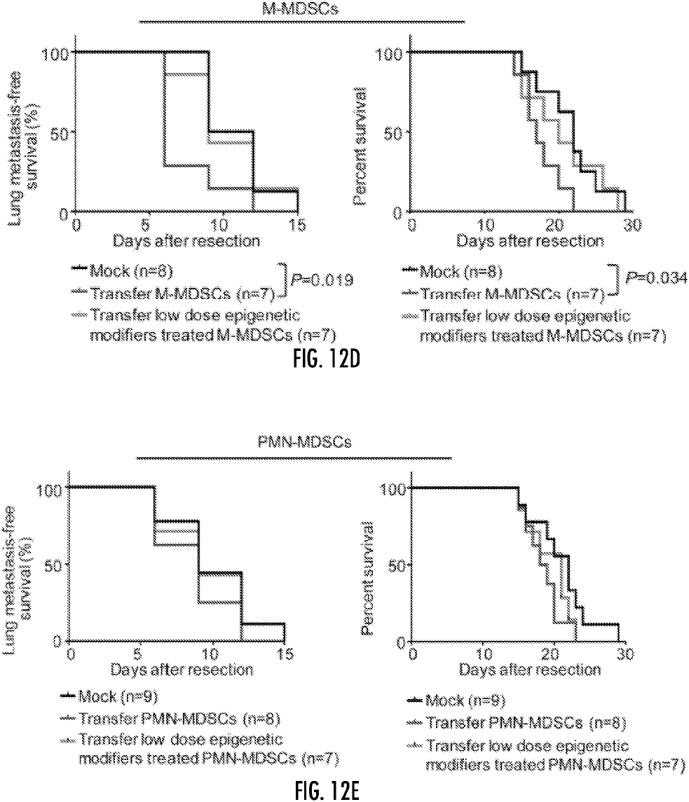

Characterization using Immunological Genome Project (ImmGen) criteria further defined the resulting populations in the lung premetastatic microenvironment.²⁸ Our top 200 upregulated genes in monocytic MDSCs from the lungs of mice treated with low-dose AET map specifically to lung interstitial macrophages (FIG. 3D). Additionally, flow cytometry shows that lung interstitial macrophages (CD11b⁺CD11c⁺CD64^{high}MHC-II⁺CD24⁻)²⁹ increase in mice treated with low-dose AET (FIGS. 12A, 12B). This observation is confirmed by adoptively transferring 5x10⁶ CD45.1⁺ monocytic MDSCs into CD45.2⁺ mice. As expected, 36 h after transfer, the CD45.1⁺ monocytic MDSCs differentiated towards a more-interstitial macrophage-like phenotype in the lungs of CD45.2 mice treated with low-dose AET (FIG. 3E, FIG. 12C). In summary, low-dose AET may skew monocytic MDSCs towards an interstitial macrophage-like phenotype in the lung premetastatic microenvironment. This acquisition of a MDSC-to-macrophage program is associated with CCR2 down regulation (a known consequence of monocyte-to-macrophage differentiation³⁰), thus also establishing the functional implications of our transcriptional data. Moreover, we found that transferring wildtype monocytic MDSCs rescued metastases in Ccr2-knockout mice and resulted in shorter overall survival times, whereas transferring wild-type polymorphonuclear MDSCs did not. By contrast, transferring wild-type monocytic MDSCs from mice treated with low-dose AET brought only limited change to both the lung metastatic rate and overall survival time of Ccr2-knockout mice (FIGS. 12D, 12E). These findings imply that the decreased accumulation of monocytic MDSCs in the premetastatic niche owing to low-dose AET is mainly dependent on CCR2 signaling.

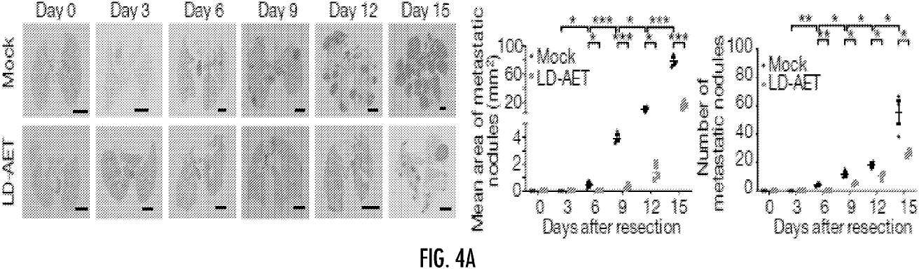

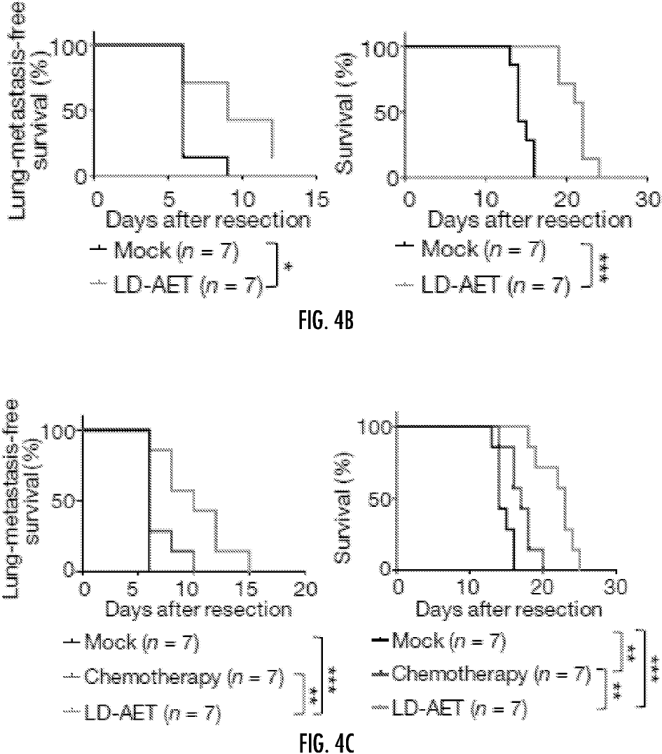

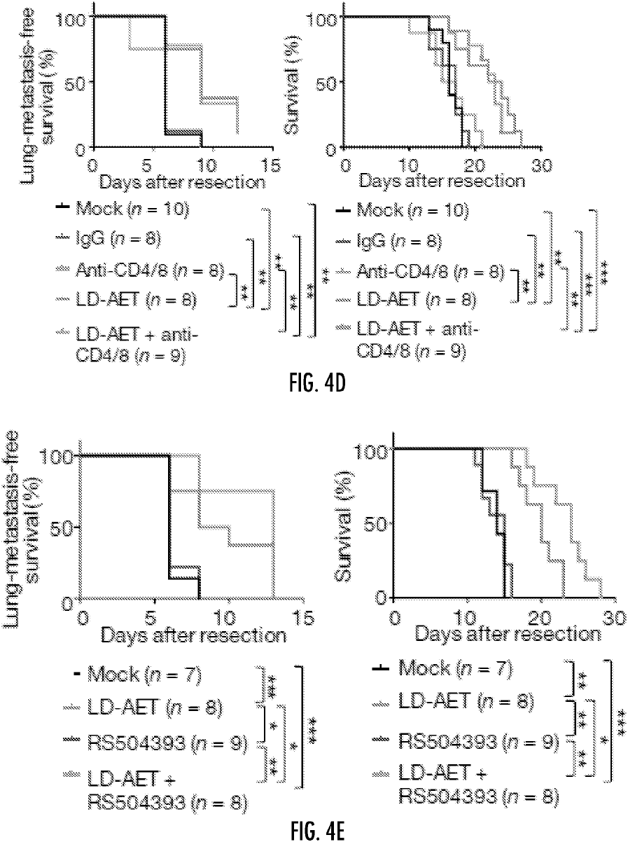

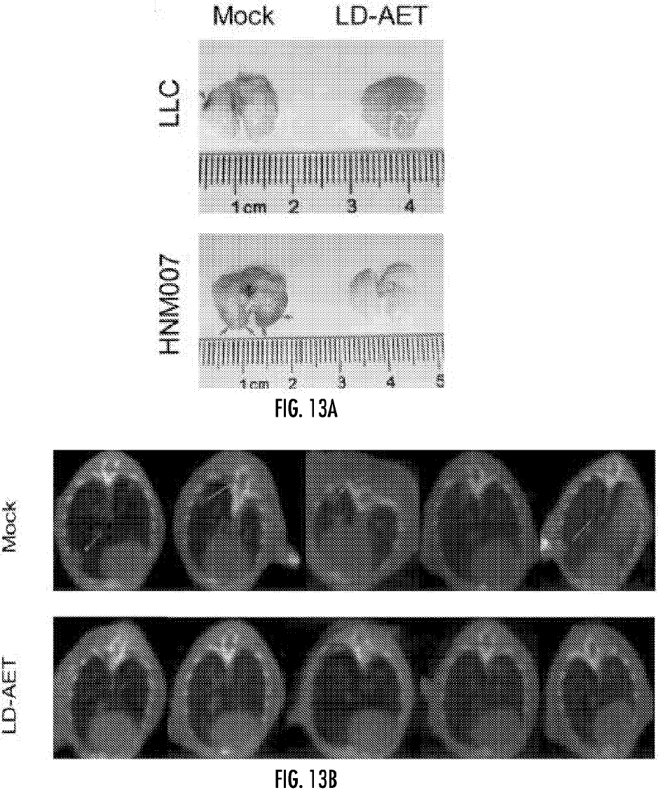

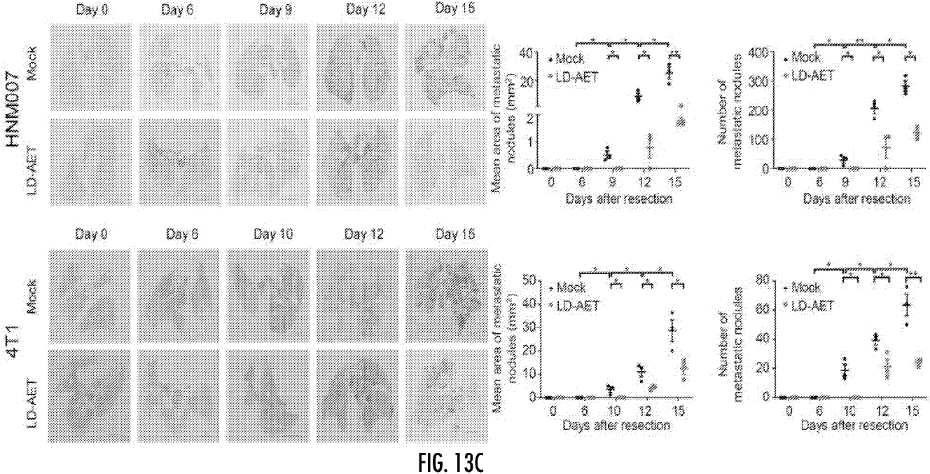

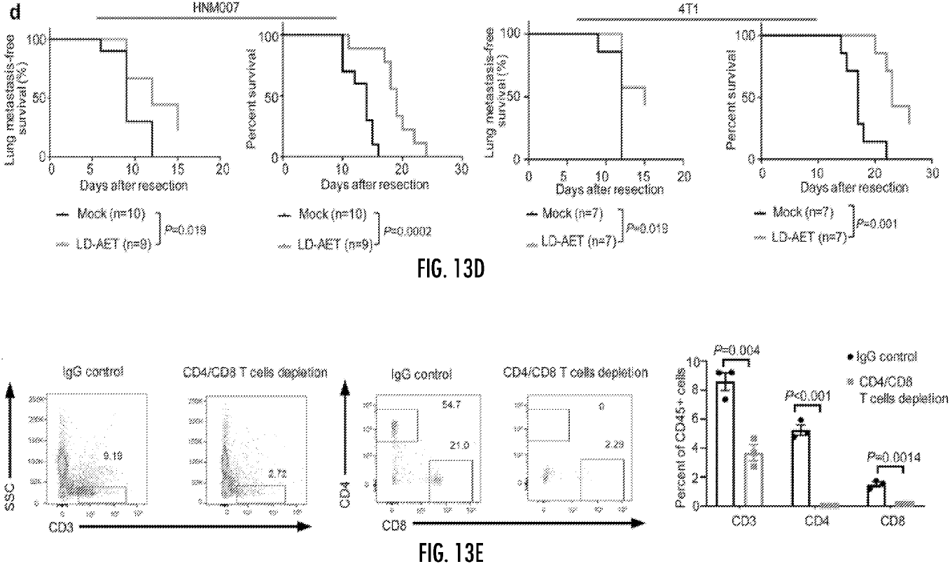

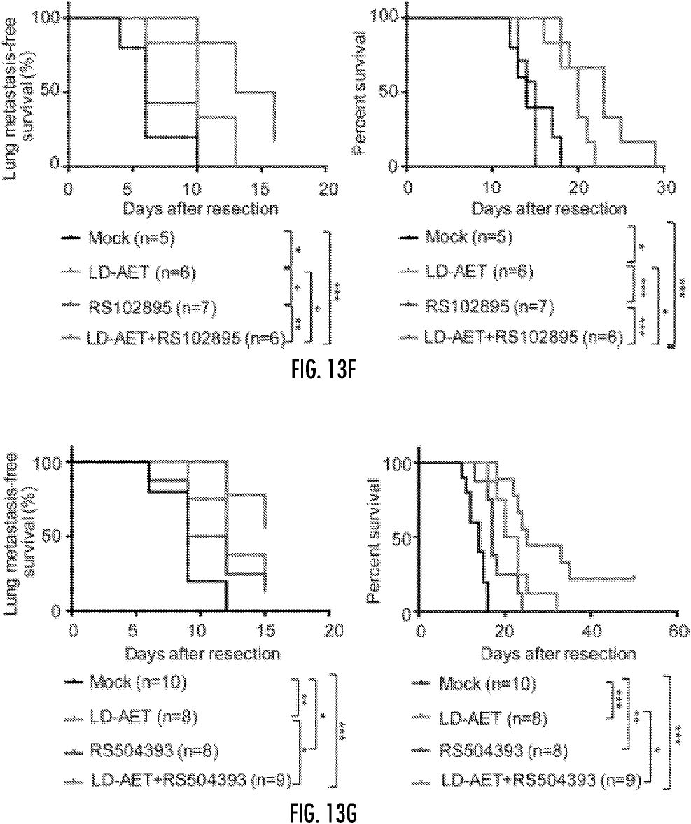

Low-dose AET increases overall survival. In our mouse models, low-dose AET reduces pulmonary metastases, as shown by H&E staining, gross pathological findings and cone-beam computed tomography imaging (FIG. 4A, FIGS. 13A-13C). In all three mouse models, low-dose AET prolongs disease-free survival and overall survival time (FIG. 4B, FIG. 13D). Compared to adjuvant chemotherapy, low-dose AET confers both a longer disease-free and overall survival in the LLC model (FIG. 4C). It has previously been found that epigenetic therapy results in a strong attraction of CD8⁺ T cells to NSCLC tumors and a reversal of the immune-exhaustion profile of these cells14. However, when we depleted CD4 and CD8 T cells (from day 1 to day 6) in our LLC model, we observed that there was no difference during low-dose AET either in disease-free or overall survival—indicating that the reduction in metastases was independent of T cells (FIG. 4D, FIG. 13E). Finally, the combination of low-dose AET and CCR2 antagonists in our LLC and HNM007 models shows synergy in disease-free and overall survival (FIG. 4E, FIGS. 13F, 13G).

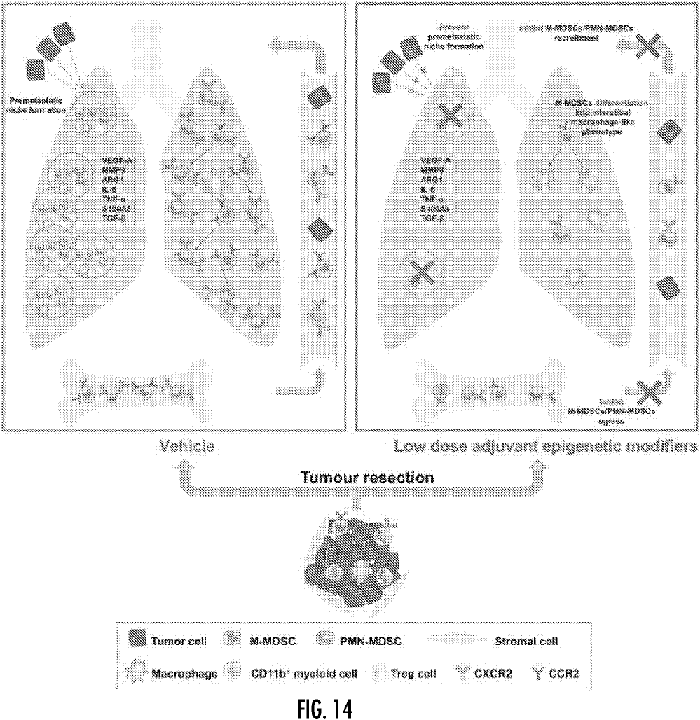

Our observation that the lung premetastatic niche persists even after resection has translational implications. First, the results stress a potential use of epigenetic treatment as an adjuvant therapy focused on the perturbation of MDSCs. Second, our findings suggest that combining low-dose AET with CCR2 antagonists may be an emerging paradigm to prevent the accumulation of MDSCs in the premetastatic niche, thus inhibiting metastases and extending survival. Third, we provide compelling evidence that if monocytic MDSCs successfully migrate to the lung premetastatic niche, epigenetic modifiers can skew the population to a more-interstitial macrophage-like phenotype, antagonizing their prometastatic functionality in that microenvironment (FIG. 14). The post-resection recurrence of early-stage cancer (especially for the tumor types studied here) is a considerable clinical challenge, and effective adjuvant therapies are lacking. Low-dose AET represents a potentially efficacious treatment to use in the absence of manifest primary tumor burden after resection. Our therapeutic paradigm may augment the efficacy of early cancer resection (which still represents the most effective treatment), while robustly inhibiting recurrence. We plan to translate these preclinical findings to a clinical trial in early-stage cancer using low-dose AET and CCR2 antagonists to prevent metastatic recurrence.

### REFERENCES

1. Mahvi, D. A., Liu, R., Grinstaff, M. W., Colson, Y. L. & Raut, C. P. Local cancer recurrence: the realities, challenges, and opportunities for new therapies. CA Cancer J. Clin. 68, 488-505 (2018).

2. Arriagada, R. et al. Long-term results of the international adjuvant lung cancer trial evaluating adjuvant cisplatin-based chemotherapy in resected lung cancer. J. Clin. Oncol. 28, 35-42 (2010).

3. Pan, H. et al. 20-year risks of breast-cancer recurrence after stopping endocrine therapy at 5 years. N. Engl. J. Med. 377, 1836-1846 (2017).

4. Kaplan, R. N. et al. VEGFR1-positive haematopoietic bone marrow progenitors initiate the pre-metastatic niche. Nature 438, 820-827 (2005).

5. Bonapace, L. et al. Cessation of CCL2 inhibition accelerates breast cancer metastasis by promoting angiogenesis. Nature 515, 130-133 (2014).

6. Peinado, H. et al. Pre-metastatic niches: organ-specific homes for metastases. Nat. Rev. Cancer 17, 302-317 (2017).

7. Steeg, P. S. Targeting metastasis. Nat. Rev. Cancer 16, 201-218 (2016).

8. Pignon, J. P. et al. Lung adjuvant cisplatin evaluation: a pooled analysis by the LACE collaborative group. J. Clin. Oncol. 26, 3552-3559 (2008).

9. Allum, W. H., Stenning, S. P., Bancewicz, J., Clark, P. I. & Langley, R. E. Long-term results of a randomized trial of surgery with or without preoperative chemotherapy in esophageal cancer. J. Clin. Oncol. 27, 5062-5067 (2009).

10. Shapiro, J. et al. Neoadjuvant chemoradiotherapy plus surgery versus surgery alone for oesophageal or junctional cancer (CROSS): long-term results of a randomised controlled trial. Lancet Oncol. 16, 1090-1098 (2015).

11. Kris, M. G. et al. Adjuvant systemic therapy and adjuvant radiation therapy for stage I to IIIA completely resected non-small-cell lung cancers: American Society of Clinical Oncology/Cancer Care Ontario clinical practice guideline update. J. Clin. Oncol. 35, 2960-2974 (2017).

12. Forde, P. M. et al. Neoadjuvant PD-1 blockade in resectable lung cancer. N. Engl. J. Med. 378, 1976-1986 (2018).

13. Postmus, P. E. et al. Early and locally advanced non-small-cell lung cancer (NSCLC): ESMO clinical practice guidelines for diagnosis, treatment and follow-up. Ann. Oncol. 28 (Suppl. 4), iv1-iv21 (2017).

14. Topper, M. J. et al. Epigenetic therapy ties MYC depletion to reversing immune evasion and treating lung cancer. Cell 171, 1284-1300.e21 (2017).

15. Liu, Y. & Cao, X. Characteristics and significance of the pre-metastatic niche. Cancer Cell 30, 668-681 (2016).

16. Kitamura, T., Qian, B. Z. & Pollard, J. W. Immune cell promotion of metastasis. Nat. Rev. Immunol. 15, 73-86 (2015).

17. Qin, H. et al. Generation of a new therapeutic peptide that depletes myeloid-derived suppressor cells in tumor-bearing mice. Nat. Med. 20, 676-681 (2014).

18. Juergens, R. A. et al. Combination epigenetic therapy has efficacy in patients with refractory advanced non-small cell lung cancer. Cancer Discov. 1, 598-607 (2011).

19. Kim, K. et al. Eradication of metastatic mouse cancers resistant to immune checkpoint blockade by suppression of myeloid-derived cells. Proc. Natl Acad. Sci. USA 111, 11774- 11779 (2014).

20. Youn, J.-I. et al. Epigenetic silencing of retinoblastoma gene regulates pathologic differentiation of myeloid cells in cancer. Nat. Immunol. 14, 211-220 (2013).

21. Qian, B.-Z. et al. CCL2 recruits inflammatory monocytes to facilitate breast-tumor metastasis. Nature 475, 222-225 (2011).

22. Sun, S. C. The non-canonical NF-_{K}B pathway in immunity and inflammation. Nat. Rev. Immunol. 17, 545-558 (2017).

23. Highfill, S. L. et al. Disruption of CXCR2-mediated MDSC tumor trafficking enhances anti-PD1 efficacy. Sci. Transl. Med. 6, 237ra67 (2014).

24. Di Mitri, D. et al. Tumor-infiltrating Gr-1⁺ myeloid cells antagonize senescence in cancer. Nature 515, 134-137 (2014).

25. Saeed, S. et al. Epigenetic programming of monocyte-to-macrophage differentiation and trained innate immunity. Science 345, 1251086 (2014).

26. Auffray, C., Sieweke, M. H. & Geissmann, F. Blood monocytes: development, heterogeneity, and relationship with dendritic cells. Annu. Rev. Immunol. 27, 669-692 (2009).

27. Miller, J. C. et al. Deciphering the transcriptional network of the dendritic cell lineage. Nat. Immunol. 13, 888-899 (2012).

28. Heng, T. S. & Painter, M. W. The immunological genome project: networks of gene expression in immune cells. Nat. Immunol. 9, 1091-1094 (2008).

29. Misharin, A. V., Morales-Nebreda, L., Mutlu, G. M., Budinger, G. R. & Perlman, H. Flow cytometric analysis of macrophages and dendritic cell subsets in the mouse lung. Am. J. Respir. Cell Mol. Biol. 49, 503-510 (2013).

30. Phillips, R. J., Lutz, M. & Premack, B. Differential signaling mechanisms regulate expression of CC chemokine receptor-2 during monocyte maturation. J. Inflamm. (Lond.) 2, 14 (2005).

31. Day, C. P., Carter, J., Bonomi, C., Hollingshead, M. & Merlino, G. Preclinical therapeutic response of residual metastatic disease is distinct from its primary tumor of origin. Int. J. Cancer 130, 190-199 (2012).

32. Yu, J. et al. Systematic study of target localization for bioluminescence tomography guided radiation therapy. Med. Phys. 43, 2619 (2016).

33. Dieckmann, D., Plottner, H., Berchtold, S., Berger, T. & Schuler, G. Ex vivo isolation and characterization of CD4⁺CD25⁺ T cells with regulatory properties from human blood. J. Exp. Med. 193, 1303-1310 (2001).

34. Subramanian, A. et al. Gene set enrichment analysis: a knowledge-based approach for interpreting genome-wide expression profiles. Proc. Natl Acad. Sci. USA 102, 15545-15550 (2005).

35. Huang, W., Sherman, B. T. & Lempicki, R. A. Bioinformatics enrichment tools: paths toward the comprehensive functional analysis of large gene lists. Nucleic Acids Res. 37, 1-13 (2009).

36. Huang, W., Sherman, B. T. & Lempicki, R. A. Systematic and integrative analysis of large gene lists using DAVID bioinformatics resources. Nat. Protocols 4, 44-57 (2009).

We claim:

1. A method for inhibiting lung cancer metastases in a post-resection patient comprising the step of administering low dose adjuvant epigenetic therapy (LDAET) to inhibit the migration of myeloid-derived suppressor cells from the bone marrow to the lung premetastatic environment.

2. The method of claim 1, wherein the LDAET comprises administering a demethylating agent.

3. The method of claim 2, wherein the demethylating agent comprises at least one of 5-azacytidine (azacytidine; AZA), 5-azadeoxycytidine (decitabine; DAC), SGI-110 (guadecitabine), zebularine and procaine.

4. The method of claim 2, wherein the demethylating agent comprises at least one of 6-Thioguanine; 5-Fluoro-2′-deoxycytidine; pseudocytidine; 5,6-Dihydro-5-azacytidine; fazarbine; 2′-Deoxy-5,6-dihydro-5-azacytidine; 4′-Thio-2′-deoxycytidine; 5-aza-4′-thio-2′-deoxycytidine; CP-4200; RX-3117; 2′-deoxy-N4-[2-(4-nitrophenyl)ethoxycarbonyl]-5-azacytidine; and 2′3′ 5′ Triacetyl-5-azacytidine.

5. The method of claim 1, wherein the LDAET comprises administering a histone deacytelase (HDAC) inhibitor.

6. The method of claim 5, wherein the HDAC inhibitor comprises at least one of givinostat, entinostat, trichostatin A (TSA), Vorinostat (SAHA), Valproic Acid (VPA), romidepsin (FK228, depsipeptide), MS-275, tucidinostat (chidamide), panobinostat (Farydak, LBH589),belinostat (13XD101), mocetinostat (MGCD0103), abexinostat (PCI-24781), SB939, resminostat (4SC-201),quisinostat (JNJ26481585), Kevetrin, CUDC-101, AR-42, CHR-2845, CHR-3996, Domatinostat (4SC-202), ricolinostat (ricolinostat (ACY-1215)),and ME-344.

7. The method of claim 5, wherein the HDAC inhibitor comprises at least one of ACY-738, BG45, Biphenyl-4-sulfonyl chloride, BRD73954, CAY10603, Citarinostat (ACY-241), CUDC-907, Dacinostat (LAQ824), Droxinostat, ITSA-1 (ITSA1), LMK-235, M344, MC1568, Nexturastat A, PCI-34051, Pracinostat (SB939), RG2833 (RGFP109), RGFP966, Santacruzamate A (CAY10683), SKLB-23bb, Sodium butyrate, Splitomicin, Suberohydroxamic acid, Tacedinaline (CI994), Tasquinimod, TH34, Tinostamustine(EDO-S101), TMP195, TMP269, Tubacin, Tubastatin A, Tubastatin A HC1, UF010, and WT161.

8. The method of claim 1, further comprising the step of administering a C-C chemokine receptor type 2 (CCR2) inhibitor.

9. The method of the claim 8, wherein the CCR2 inhibitor comprises at least one of CCX140, CCX872, PF-04136309 (PF-6309), PF-04178903, INCB-8696, CCX-915, MLN-1202, JNJ-17166864; AZD-2423, INCB-003284, BMS-741672, MK-0812; PF-04634817, CNTO888, and 747 (kaempferol 3-(2,4-di-E-p-coumaroylrhamnoside).

10. The method of claim 1, further comprising the step of administering a C-X-C motif chemokine receptor 2 (CXCR2) inhibitor.

11. The method of claim 10, wherein the CXCR2 inhibitor comprises at least one of AZD5069, MK-7123 (SCH527123, Navarixin), SB-332235, danirixin, elubrixin, PS-291822, SB225002, SX-682, SX-576, SX-517, ladarixin, reparixin, reparixin L-lysine salt, DF2755A, CXCL8 fragment comprising amino acids 3-74 and substitutions K11R/G31P (G31P); DF2162 and SCH-479833.

12. A method for inhibiting cancer metastases in a post-resection patient comprising the step of administering LDAET to inhibit the migration of myeloid-derived suppressor cells from the bone marrow to the premetastatic environment.

13. The method of claim 12, wherein the LDAET comprises one or more of epigenetic therapy, a CCR2 inhibitor and a CXCR2 inhibitor.

14. The method of claim 13, wherein the epigenetic therapy comprises at least one of a demethylating agent and an HDAC inhibitor.

15. The method of claim 13, wherein the epigenetic therapy comprises nucleoside analogs that target DNA methyltransferases.

16-20. (canceled)
